(19) United States
(12) Patent Application Publication
(10) Pub. No.: US 2005/0175584 A1
(43) Pub. Date: Aug. 11, 2005

(54) Functionalized colloidal metal compositions and methods

(76) Inventors: Giulio F. Paciotti, Baltimore, MD (US); Lawrence Tamarkin, Rockville, MD (US)

Correspondence Address: JOHN S. PRATT, ESQ;KILPATRICK STOCKTON, LLP, 1100 PEACHTREE STREET, ATLANTA, GA, 30309, US

(21) Appl. No.: 11/046,204

(22) Filed: Jan. 28, 2005

Related U.S. Application Data
(60) Provisional application No. 60/540,075, filed on Jan. 28, 2004.

(51) Int. Cl. A61K 48/00; A61K 38/20; A61K 38/19; A61K 38/17; C07K 14/54; C07K 14/47; C07H 21/04

(52) U.S. Cl. 424/85.2; 514/44; 514/6; 424/85.4; 530/351; 530/400; 536/23.1

(57) ABSTRACT

The present invention comprises methods and compositions for making functionalized/reactive colloidal metal compositions and the uses thereof. The present invention comprises compositions and methods for delivery systems of agents, including therapeutic compounds, pharmaceutical agents, drugs, detection agents, nucleic acid sequences and biological factors. In general, these vector compositions comprise a functionalized/reactive colloidal metal, and an agent. The invention also comprises methods and compositions for the treatment of cancer.

### CROSS REFERENCE TO RELATED APPLICATIONS

This application claims the benefit of priority to U.S. Provisional Application No. 60/540,075, filed Jan. 28, 2004.

### FIELD OF THE INVENTION

The present invention relates to colloidal metal compositions and methods for making and using such compositions. In general, the present invention relates to compositions and methods for generalized delivery of agents and delivery of agents to specific sites.

### BACKGROUND OF THE INVENTION

It has long been a goal of therapeutic treatment to find the magic bullet that would track to the site of need and deliver a therapeutic response without undue side effects. Many approaches have been tried to reach this goal. Therapeutic agents have been designed to take advantage of differences in active agents, such as hydrophobicity or hydrophilicity, or size of therapeutic particulates for differential treatment by cells of the body. Therapies exist that deliver therapeutic agents to specific segments of the body or to particular cells by in situ injection, and either use or overcome body defenses such as the blood-brain barrier, that limit the delivery of therapeutic agents.

One method that has been used to specifically target therapeutic agents to specific tissues or cells is delivery based on the combination of a therapeutic agent and a binding partner of a specific receptor. For example, the therapeutic agent may be cytotoxic or radioactive and when combined with a binding partner of a cellular receptor, cause cell death or interfere with genetic control of cellular activities once bound to the target cells. This type of delivery device requires having a receptor that is specific for the cell-type to be treated, an effective binding partner for the receptor, and an effective therapeutic agent. Molecular genetic manipulations have been used to overcome some of these problems.

An important and desired target for delivery of specific agents is the immune system. The immune system is a complex response system of the body that involves many different kinds of cells that have differing activities. Activation of one portion of the immune system usually causes a variety of responses due to unwanted activation of other related portions of the system. Currently, there are no satisfactory methods or compositions for producing a specifically desired response by targeting the specific components of the immune system.

The immune system is a complex interactive system of the body that involves a wide variety of components, including cells, and cellular factors, which interact with stimuli from both inside the body and outside the body. Aside from its direct action, the immune system's response is also influenced by other systems of the body including the nervous, respiratory, circulatory, and digestive systems.

One of the better-known aspects of the immune system is its ability to respond to foreign antigens presented by invading organisms, cellular changes within the body, or from vaccination. Some of the first kinds of cells that respond to such activation of the immune system are phagocytes and natural killer cells. Phagocytes include among other cells, monocytes, macrophages, and polymorphonuclear neutrophils. These cells generally bind to the foreign antigen, internalize it and often times destroy it. They also produce soluble molecules that mediate other immune responses, such as inflammatory responses. Natural killer cells can recognize and destroy certain virally-infected embryonic and tumor cells. Other factors of the immune response include complement pathways, which are capable of responding independently to foreign antigens or acting in concert with cells or antibodies.

One of the aspects of the immune system that is important for vaccination is the specific response of the immune system to a particular pathogen or foreign antigen. Part of the response includes the establishment of “memory” for that foreign antigen. Upon a secondary exposure, the memory function allows for a quicker and generally greater response to the foreign antigen. Lymphocytes in concert with other cells and factors play a major role in both the memory function and the response.

Generally, it is thought that the response to antigens involves both humoral responses and cellular responses. Humoral immune responses are mediated by non-cellular factors that are released by cells and which may or may not be found free in the plasma or intracellular fluids. A major component of a humoral response of the immune system is mediated by antibodies produced by B lymphocytes. Cell-mediated immune responses result from the interactions of cells, including antigen presenting cells and B lymphocytes (B cells) and T lymphocytes (T cells).

One of the most widely employed aspects of the immune response capabilities is the production of monoclonal antibodies. The advent of monoclonal antibody (Mab) technology in the mid 1970s provided a valuable new therapeutic and diagnostic tool. For the first time, researchers and clinicians had access to unlimited quantities of uniform antibodies capable of binding to a predetermined antigenic site and having various immunological effector functions. Currently, the techniques for production of monoclonal antibodies are well known in the art.

Vaccines may be directed at any foreign antigen, whether from another organism, a changed cell, or induced foreign attributes in a normal “self” cell. The route of administration of the foreign antigen can help determine the type of immune response generated. For example, delivery of antigens to mucosal surfaces, such as oral inoculation with live polio virus, stimulates the immune system to produce an immune response at the mucosal surface. Injection of antigen into muscle tissue often promotes the production of a long lasting IgG response.

Vaccines may be generally divided into two types, whole and subunit vaccines. Whole vaccines may be produced from viruses or microorganisms which have been inactivated or attenuated or have been killed. Live attenuated vaccines have the advantage of mimicking the natural infection enough to trigger an immune response similar to the response to the wild-type organism. Such vaccines generally provide a high level of protection, especially if administered by a natural route, and some may only require one dose to confer immunity. Another advantage of some attenuated vaccines is that they provide person-to-person passage among members of the population. These advantages, however, are balanced with several disadvantages. Some attenuated vaccines have a limited shelf-life and cannot withstand storage in tropical environments. There is also a possibility that the vaccine will revert to the virulent wild-type of the organism, causing harmful, even life-threatening, illness. The use of attenuated vaccines is contraindicated for immunodeficient states, such as AIDS, and in pregnancy.

Killed vaccines are safer in that they cannot revert to virulence. They are generally more stable during transport and storage and are acceptable for use in immunocompromised patients. However, they are less effective than the live attenuated vaccines, usually requiring more than one dose. Additionally, they do not provide for person-to-person passage among members of the population.

Production of subunit vaccines requires knowledge about the epitopes of the microorganism or cells to which the vaccine should be directed. Other considerations in designing subunit vaccines are the size of the subunit and how well the subunit represents all of the strains of the microorganism or cell. The current focus for development of bacterial vaccines has shifted to the generation of subunit vaccines because of the problems encountered in producing whole bacterial vaccines and the side effects associated with their use. Such vaccines include a typhoid vaccine based upon the Vi capsular polysaccharide and the Hib vaccine to Haemophilus influenzae.

Because of the safety concerns associated with the use of attenuated vaccines and the low efficacy of killed vaccines, there is a need in the art for compositions and methods that enhance vaccine efficacy. There is also a need in the art for compositions and methods of enhancing the immune system, which stimulate both humoral and cell-mediated responses. There is a further need in the art for the selective adjustment of an immune response and manipulating the various components of the immune system to produce a desired response. Additionally, there is a need for methods and compositions that can accelerate and expand the immune response for a more rapid activation response. There is an increased need for the ability to vaccinate populations, of both humans and animals, with vaccines that provide protection with just one dose.

What is needed are compositions and methods to target the delivery of specific agents to only the target cells. Such compositions and methods should be able to deliver therapeutic agents to the target cells efficiently. What is also needed are compositions and methods that can be used both in in vitro and in vivo systems.

Simple, efficient delivery systems for delivery of specific therapeutic agents to specific sites in the body for the treatment of diseases or pathologies or for the detection of such sites are not currently available. For example, current treatments for cancer include administration of chemotherapeutic agents and other biologically active factors such as cytokines and immune factors that impact the entire organism. The side effects include organ damage, loss of senses such as taste and feel, and hair loss. Such therapies provide treatment for the condition, but also require many adjunct therapies to treat the side effects.

What is needed are compositions and methods for delivery systems of agents that effect the desired cells or site. These delivery systems could be used for delivery to specific cells of agents of all types, including detection, therapeutic agents, prophylaxis and synergistic agents. What is also needed are delivery systems that do not cause unwanted side effects in the entire organism. Furthermore, what are needed are compositions that are stable under a variety of physiological conditions, including pH and in the presence of salt.

### SUMMARY OF THE INVENTION

The present invention comprises compositions and methods for delivery systems of agents, including, but not limited to, therapeutic compounds, pharmaceutical agents, drugs, detection agents, nucleic acid sequences, antigens, enzymes and biological factors. In general, these vector compositions comprise a functionalized/reative colloidal metal sol, which is linked to the agent to be delivered.

In one embodiment, preferred compositions of the present invention comprise vectors comprising colloidal metal sols, preferably gold metal sols, associated with derivatized-PEG, preferably thiol-PEG (PEG(SH)ₙ), or derivatized poly-1-lysine, preferably poly-1-lysine thiol (PLL(SH)ₙ) and may also comprise one or more agents that aid in specific targeting of the vector or have therapeutic effects or can be detected.

In an alternative embodiment, preferred compositions comprise modification of the agent to incorporate a free sulfhydryl/thiol group, which is then linked to/incorporated into the functionalized/reactive colloidal metal sol. The agent, the colloidal metal or both may be modified to incorporate reactive groups, preferably thiol groups that facilitate binding.

The present invention further comprises compositions and methods for making functionalized/reactive colloidal metal sols using derivatized thiol or derivatized poly-1-lysine as reducing agents. The use of derivatized thiol or derivatized poly-1-lysine incorporates the thiol groups onto the surface of the colloidal metal.

In another embodiment, the present invention comprises a method for making functionalized/reactive colloidal metal sols using derivatized PEG thiol, derivatized poly-1-lysine or alkane thiol as a reducing agent.

The present invention further comprises methods of delivery by administering the compositions of the invention by known methods such as injection or orally, wherein the compositions are delivered to specific cells or organs. It is an aspect of the invention that the route of administration is not considered critical for the effective delivery of the composition. It is anticipated that one of ordinary skill in the art would be capable of establishing an appropriate route of administration to achieve the required objective. Due to their dimensional similarities to macromolecules colloidal metal conjugates are particularly useful in detection and imaging procedures and as long term carriers for drug release or drug delivery. In one embodiment, the present invention comprises methods for treating diseases, including, but not limited to, cancer or solid tumors, by administering the compositions of the present invention comprising agents that are known for the treatment of such diseases. Another embodiment comprises vector compositions comprising derivatized PEG, TNF (Tumor Necrosis Factor) and anti-cancer agents, associated with functionalized/reactive colloidal metal particles. A further embodiment comprises derivatized poly-1-lysine and therapeutic agents, associated with colloidal metal particles. In another embodiment, the present invention comprises methods for gene therapy by administering the compositions of the present invention comprising agents that are used for gene therapy, such as oligonucleotides, antisense oligonucleotides, vectors, ribozymes, DNA, RNA, sense oligonucleotides, interference RNA (RNAi) and nucleic acids.

The present invention also comprises methods and compositions suitable for lyophilizing so that the compositions have a long shelf life and can be easily transported.

### BRIEF DESCRIPTION OF THE DRAWINGS

FIG. 1 is a schematic of the modification of the surface of a colloidal gold nanoparticle by alkane thiols.

FIG. 2 is a schematic of the generation of functionalized colloidal gold particles using a bi-functional reducing agent.

FIG. 3A is a schematic of a 4-arm PEG-THIOL (PEG(SH)₄).

FIG. 3B is a schematic of the thiolation of poly-1-lysine by 2-iminothiolane.

FIG. 4 is a chart of particle size characterization of functionalized colloidal gold nanoparticles synthesized with 2 ml (A) or 1 ml (B) of the 4-arm PEG-THIOL moiety.

FIG. 5 is a chart of particle size characterization of functionalized colloidal gold nanoparticles synthesized with thiolate poly-1-lysine polymer containing a 5 mol (A) or 2 mol (B) excess of thiol.

FIG. 6 is gel of PLL(SH)₅ functionalized colloidal gold binding of plasmid DNA.

FIG. 7 is a schematic of the apparatus to bind an agent to functionalized colloidal metal nanoparticles.

FIG. 8 is a table of representative functional groups on both the functionalized colloidal metal and the agent.

### DETAILED DESCRIPTION

The present invention may be understood more readily by reference to the following detailed description of specific embodiments included herein. Although the present invention has been described with reference to specific details of certain embodiments, thereof, it is not intended that such details should be regarded as limitations upon the scope of the invention. The text of the references mentioned herein are hereby incorporated by reference in their entirety, including U.S. Provisional Application Ser. No. 60/540,075.

The present invention comprises improved methods comprising the use of reducing agents for making functionalized colloidal metal sols. In one embodiment the invention comprises compositions and methods for making functionalized colloidal metal sols, using derivatized thiol or derivatized poly-Amino-acid as reducing agents, thereby incorporating the thiol groups in the colloidal metal particles during formation. The present invention also contemplates using polyethylene glycol (PEG)-thiol or thiolated poly-1-lysine as reducing agents for making functionalized colloidal metal sols. Other reducing agents known to those skilled in the art are contemplated to be within the scope of the present invention.

The present invention further comprises methods for making the compositions and administering the compositions in vitro and in vivo. In general, the present invention contemplates compositions comprising metal sol particles associated with any or all of the following components alone or in combinations: biologically active agents, therapeutic agents, pharmaceutical agents, drugs, detection agents, nucleic acid sequences, targeting molecules, integrating molecules, biological factors and one or more types of polyethylene gycol (PEG), derivatized PEG, poly-1-lysine or derivatized poly-1-lysine. Additionally, the agents may be modified, such as by incorporating a free sulfhydryl/thiol group which are then linked to/incorporated into the colloidal metal sol.

As used herein the terms “colloidal metal”, “functionalized/reactive colloidal metal particles”, “functionalized nanoparticles”, or “reactive metal sol” are used interchangeably to define functionalized/reactive colloidal metal particles that are formed upon exposure to a reducing agent, comprising derivatized thiol, derivatized poly-amino acid and the like as determined by one of ordinary skill in the art. Unless explicitly stated, or unless the context dictates otherwise, these terms do not refer to nanoparticles formed using sodium citrate as a reducing agent (i.e. the Frens method).

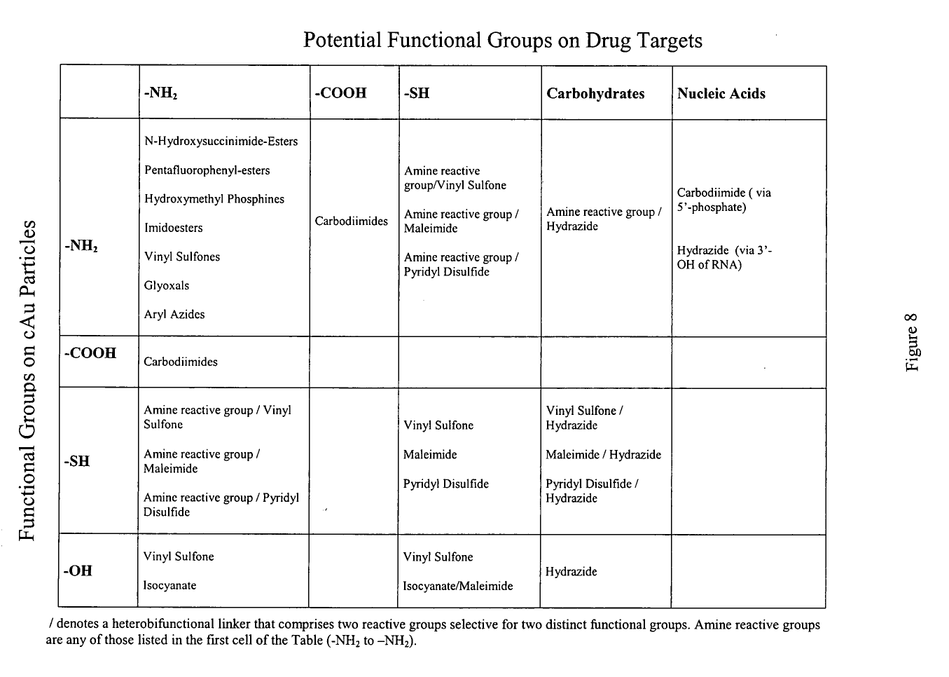

It will be apparent to one of ordinary skill in the art that the functionalized/reactive colloidal metal particles may comprise additional functional or reactive groups on or attached to the surface of the functionalized/reactive colloidal metal particle. As such these additional reactive or functional groups may act as sites for the attachment of agents. FIG. 8 provides an exemplary and non-limiting representative table of functional groups that may be present on or attached to the functionalized colloidal metal or agent. Furthermore, this table provides representative heterobifunctional linkers that comprise at least two reactive groups selective for two distinct functional groups.

It is contemplated by the instant invention that one of ordinary skill in the art would be capable of modifying the reducing agent to allow additional functional or reactive groups to be applied to or attached to the surface of the functionalized/reactive colloidal particle. It is proposed that additional functional groups may be incorporated onto or attached to the functionalized/reactive colloidal metal particle through the modulation of the reducing agent. It is also envisaged that one of ordinary skill in that art may modulate the functionality of a reducing agent which comprises a polymer. For example, thiolated poly-amino acids, such as poly-1-lysine or poly-glutamic acid may be utilized as reducing agents to add specific types of functional groups to the functionalized/reactive colloidal metal particle.

In one embodiment of the instant invention a derivatized thiol comprising a free thiol group and a polymer may be used to form functionalized/reactive metal particles. In another embodiment, the functionality of the above polymer may be modified to incorporate other functional groups onto to the functionalized/reactive colloidal particle, including, but not limited to, the functional groups presented in FIG. 8. In another embodiment of the instant invention carboxyl, hydroxyl and amine groups may be incorporated onto or attached to the functionalized colloidal metal particles.

The agent, the colloidal metal sol or both may be modified to incorporate reactive groups, such as those shown in FIG. 8. in some instances, thiol groups may be used to facilitate binding.

The functionalized colloidal metal sols of the present invention may be used for the delivery of agents for detection or treatment of specific cells or tissues. The delivery of agents may also be used for treatments of biological conditions, including, but not limited to, chronic and acute diseases, maintenance and control of the immune system and other biological systems, infectious diseases, vaccinations, hormonal maintenance and control, cancer, solid tumors and angiogenic states. Such delivery may be targeted to specific cells or cell types, or the delivery may be less specifically provided to the body, in methods that allow for delivery of the agent or agents in a nontoxic manner. Descriptions and uses of metal sol compositions are taught in U.S. Pat. Nos. 6,274,552, 6,407,218, 6,528,051; and related patent applications, U.S. patent application Ser. Nos. 09/808,809, 09/189,657, 10/135,886, 10,325,485, 10,672,144, 11/004,623, and 09/803,123; and U.S. Provisional Patent Applications 60/287,363, all of which are hereby incorporated by reference in their entirety.

The compositions of the invention preferably comprise a colloidal metal sol, derivatized compounds and one or more agents or modified agents. The agents may comprise biologically active agents that can be used in therapeutic applications or the agents may be useful in detection and/or imaging methods. In additional embodiments, one or more agents are admixed, associated with or bound directly or indirectly to the colloidal metal. Admixing, associating and binding includes covalent and ionic bonds and other weaker or stronger associations that allow for long term or short term association of the derivatized-PEG or the derivatized poly-1-lysine, agents, and other components with each other and with the metal sol particles.

In yet another embodiment, the compositions may optionally comprise one or more targeting molecules admixed, associated with or bound to the colloidal metal. The targeting molecule can be bound directly or indirectly to the metal particle. Indirect binding includes binding through molecules such as integrating molecules or any association with a molecule that binds to both the targeting molecule and either the metal sol or another molecule bound to the metal sol.

Of particular interest are detection agents such as dyes, molecular tagging molecules or radioactive materials that can be used for visualizing or detecting the sequestered colloidal metal vectors. Fluorescent, chemiluminescent, heat sensitive, opaque, beads, magnetic and vibrational materials are also contemplated for use as detectable agents that are associated or bound to colloidal metals in the compositions of the present invention.

Any metal salt can be used in the present invention. The term “metal”, as used herein, includes any water-insoluble metal particle or metallic compound dispersed in liquid or water, a hydrosol or a metal sol. Examples of metals, salts which can be used in the present invention include, but are not limited to, metals in groups IA, IB, IIB and IIIB of the periodic table, as well as the transition metals, especially those of group VIII. Preferred metal salts include gold, silver, aluminum, ruthenium, zinc, iron, nickel and calcium. Other suitable metal salts also include the following in all of their various oxidation states: lithium, sodium, magnesium, potassium, scandium, titanium, vanadium, chromium, manganese, cobalt, copper, gallium, strontium, niobium, molybdenum, palladium, indium, tin, tungsten, rhenium, platinum, and gadolinium. The metal salts are preferably provided in ionic form, derived from an appropriate metal compound, for example the Al³⁺, Ru³⁺, Zn²⁺, Fe³⁺, Ni²⁺ and Ca²⁺ ions.

Another preferred metal salt is gold, particularly in the form of Au³⁺. An especially preferred form of colloidal gold is HAuCl₄ (OmniCorp, South Plainfield, N.J.). Colloidal gold is comprised of nanoparticles of Au⁰ that are kept in suspension by an inherent negative surface charge that causes the particles to repel one another. In 1857 Michael Faraday manufactured the first nano-sized particles of Au⁰ by reducing gold chloride with sodium citrate. (Faraday, Philos. Trans. R. Soc. London 14:1145,1857). Frens (Frens, Nature Phys. Sci. 241: 20-22, 1972) and Horisberger (Biol. Cellulaire 36:253-258, 1979) elaborated on his discovery by demonstrating that the gold to citrate ratio controlled the size of the nanoparticles. In another embodiment, derivatized thiol or derivatized poly-amino acid may be used as reducing agents. In a preferred embodiment, PEG-thiol or thiolated poly-1-lysine may be used as the reducing agent.

A colloid is a homogenous dispersion of particles in a solution that do not settle or precipitate out of solution readily. The colloid is stabilized by electric charges on its surface due to adsorbed ions. The surface charge causes the particles to repel each other. Formulation of nanoparticles is typically observed as a three-step process: nucleation, particle growth and coagulation.

Nucleation is the formation of nuclei upon which particle growth can occur. The production of nuclei occurs through a redox reaction. Historically, this process has relied upon the oxidation of the citrate ion to yield a reducing reagent for the gold, acetone dicarboxylic acid. A type of polymerization “complexation” occurs in which the gold ions coordinate with the acetone dicarboxylic acid and join together. When the “polymer” or complex reaches a critical mass, that is just greater than its thermodynamic stability, reduction to metallic gold occurs, yielding the nuclei.

Particle growth is the addition of more gold to the existing nuclei. This process ceases when all of the gold is utilized. Creation of larger gold particles requires a coagulation of multiple nuclei. As reported by Frens and Horisberger, modulation of the sodium citrate to gold ratio resulted in particles of various sizes. Thus, control of the coagulation process during preparation determines the size, structure, and size distribution of the particles. Once the preparation of the gold nanoparticles is complete, the absence of coagulation insures its stability.

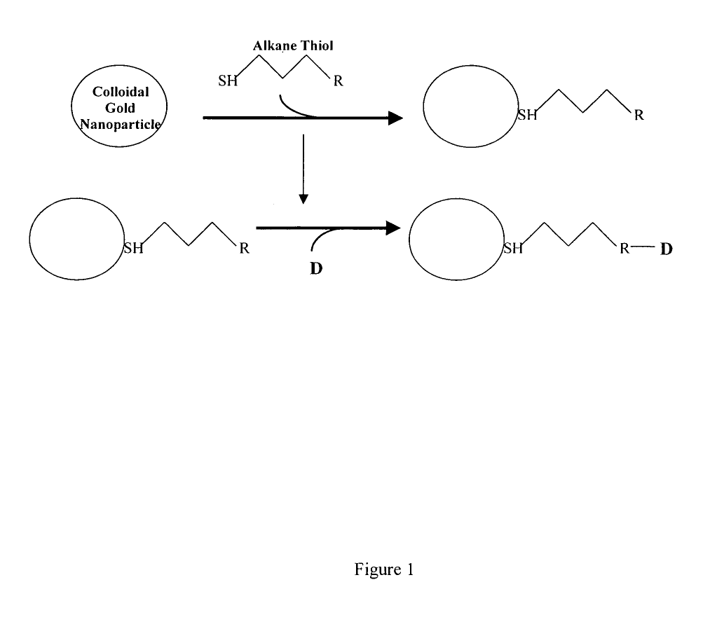

In one embodiment, the colloidal gold particles have a negative charge at an approximately neutral pH. It is thought that this negative charge prevents the attraction and attachment of other negatively charged molecules. In contrast, positively charged molecules are attracted to and bind to the colloidal gold particle. The inherent negative surface charge of colloidal gold maintains the particles in a sol state. However, cations, typically present in salt solutions, neutralize this charge and cause the particles to agglomerate and precipitate from the sol. In addition, biological molecules, such as proteins that are adsorbed to the particles' surface also negate the surface charge of the particles. This problem of agglomeration and precipitation has been overcome in the present invention by modifying either the agent attached or the colloidal metal sol, preferably the colloidal gold sol. In modifying the agents attached, the addition of a derivative such as a thiol group to the agent allows the agent to form a dative bond with the colloidal gold sol. In modifying the colloidal gold particle surface, alkane thiols are used during particle synthesis to form a bi-functional cross-linker between the colloidal particle and the agent since the thiol group serves to link the alkane thiol to the surface of the particle while the reactive group acts as an acceptor molecule for the attachment of the agent (See FIG. 1).

An alternative method for developing functionalized gold nanoparticles is known in the art. Briefly a functionalizing polymer containing a free thiol group is added during particle formation. However, particle formation still occurs by the previously described Frens methods and requires reduction of gold chloride by a separate agent such as NaBH₄. Thus, the particle reducing agent and the agent used to functionalize the gold particles are different entities.

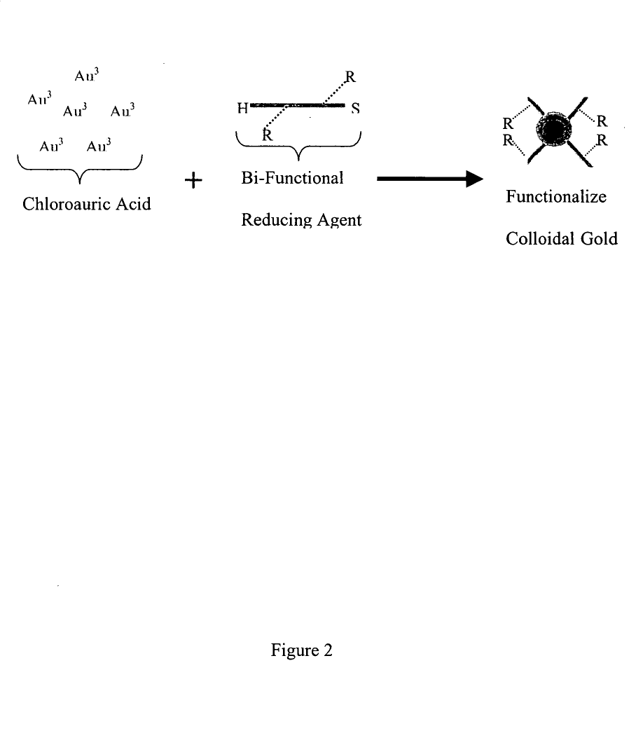

In one embodiment of the present invention, derivatized thiol or derivatized poly-1-lysine may be used as reducing agents to manufacture the gold particles (See FIG. 2). The use of derivatized thiol or derivatized poly-1-lysine incorporates the thiol groups into the colloidal gold particles. While not wishing to be bound by the following theory, it is currently theorized that the presence of the thiol groups function to initiate particle synthesis by reducing gold chloride to gold nuclei. Unlike traditional colloidal gold sols formed by the Frens method, these sols are completely stable when exposed to salts.

The formation of colloidal gold nanoparticles via the Frens/Horisberger method occurs in distinct stages. Particle nucleation is initiated immediately after the addition of sodium citrate and is observed by a change in color of the gold chloride solution from yellow to a near clear solution. After nucleation, the extent of particle growth and coagulation result in a series of further color changes. Nanoparticle solutions are well documented to undergo black, brown, and finally red coloration. This process represents a number of fragmentation reactions, which result in the formation of progressively smaller particles. A black solution may represent super-aggregates that in turn become a brown solution representing large particles, with mono-dispersed or individual colloidal gold particles appearing as a red solution.

Interestingly, the present invention does not duplicate the above reaction. Similar to the Frens preparation, a solution of gold chloride upon exposure to a bifunctional reducing agent undergoes a color change from yellow to clear solution. These data suggest that similar to the Frens preparation the functionalized/reactive gold particles of the instant invention form by a nucleation reaction. However, subsequent to the nucleation step, particle growth appears to occur by a different mechanism. Unlike the black/brown/red color described by Frens, the solution of the instant invention appeared faint red/orange initially after nucleation. With time the intensity of the color increased and became stable. This observation suggests a potential difference in the formation of the Frens nanoparticles and the instant nanoparticles. While not wishing to be bound by the following theory, it is currently theorized that the red color observed after nucleation supports the formation of individual gold nuclei formed by the bifunctional reducing agent and that these remained mono-dispersed during particle growth, thus preventing agglomeration and coagulation of particles as observed by the Frens method.

The instant application describes the use of bifunctional reducing agents to generate functionalized/reactive colloidal metal particles. An advantageous aspect of the instant invention is that functional groups present on the surface of the colloidal metal particle may be used as linking sites to attach agents that would not ordinarily bind to the functionalized/reactive colloidal metal particle. In the following non-limiting example and for clarity purposes only, the methods disclosed refer to colloidal gold. Briefly, a bifunctional reducing agent is used to generate a gold particle and place a functional group on the gold particle surface. In this instance, the reducing agent comprises a core molecule/polymer of straight or branched configuration. The reducing agent further comprises a free thiol group and a reactive group. The thiol group acts to donate its electrons to reduce cholorauric acid into clusters of gold atoms. These gold nuclei act as a platform by which particle growth can occur. While not wishing to be bound by the following theory, it is currently theorized that as the gold particles grow in size, the core molecule/polymer is embedded into the structure of the gold particles. The presence of the core molecule/polymer on the surface of the gold particles serves to stabilize the gold particles against salt precipitation. Furthermore, the functionalized/reactive colloidal gold particle allow for the binding of agents that would not ordinarily bind directly to the gold particle.

The colloidal gold is employed in the form of a sol, which contains gold particles having a range of particle sizes, preferably from about 1 to about 100 nanometers. In another embodiment, the particle size comprises about 1 nanometer to about 60 nanometers, although a preferred size is a particle size of approximately 20 to 40 nanometers. Such metal ions may be present in the sol alone or with other inorganic ions.

Another preferred metal salt is silver, particularly in a sodium borate buffer, having the concentration of between approximately 0.1% and 0.001%, and most preferably, approximately a 0.01% solution. Preferably, the color of such a colloidal silver solution is yellow and the colloidal particles range in size from 1 to 100 nm. In another embodiment, the particle size comprises about 1 nanometer to about 60 nanometers, although a preferred size is a particle size of approximately 20 to 40 nanometers. Such metal ions may be present in the complex alone or with other inorganic ions. Colloidal silver may be similarly modified with the addition of thiol groups.

The agent of the present invention can be any compound, chemical, therapeutic agent, pharmaceutical agent, drug, biological factor, enzyme, antigen, fragments of biological molecules such as antibodies, proteins, lipids, nucleic acids or carbohydrates; nucleic acids, antibodies, proteins, lipids, nutrients, cofactors, nutriceuticals, anesthetic, detection agents or an agent that has an effect in the body. Such detection and therapeutic agents and their activities are known to those of ordinary skill in the art. Additionally, the agents may be modified to include free sulfhydryl/thiol groups. In instances where thiolation adversely affects the function of the drug, secondary methods for linking the agent to the colloidal particles are required. The instant invention overcomes this problem through the incorporation of functional groups including, but not limited to, alkane thiols, onto the surface of the colloidal particle surface that facilitate the attachment of the drug to the particle.

The following are non-limiting examples of some of the agents that can be used in the present invention. One type of agent that can be employed in the present invention includes biological factors including, but not limited to, cytokines, growth factors, fragments of larger molecules that have therapeutic activity, neurochemicals, and cellular communication molecules. Examples of such agents include, but are not limited to, Interleukin-1 (“IL-1”), Interleukin-2 (“IL-2”), Interleukin-3 (“IL-3”), Interleukin-4 (“IL-4”), Interleukin-5 (“IL-5”), Interleukin-6 (“IL-6”), Interleukin-7 (“IL-7”), Interleukin-8 (“IL-8”), Interleukin- 10 (“IL-10”), Interleukin-11 (“IL-11”), Interleukin-12 (“IL-12”), Interleukin-13 (“IL-13”), Interleukin-15 (“IL-15”), Interleukin-16 (“IL-16”), Interleukin-17 (“IL-17”), Interleukin-18 (“IL-18”), lipid A, phospholipase A2, endotoxins, staphylococcal enterotoxin B and other toxins, Type I Interferon, Type II Interferon, Tumor Necrosis Factor (“TNFα or β”), Transforming Growth Factor-α (“TGF-α”), Lymphotoxin, Migration Inhibition Factor, Granulocyte-Macrophage Colony-Stimulating Factor (“CSF”), Monocyte-Macrophage CSF, Granulocyte CSF, vascular epithelial growth factor, Angiogenin, transforming growth factor-β (“TGF-β”), carbohydrate moieties of blood groups, Rh factors, fibroblast growth factor, and other inflammatory and immune regulatory proteins, nucleotides, DNA, RNA, mRNA, sense, antisense, cancer cell specific antigens; such as MART, MAGE, BAGE, and heat shock proteins (HSPs); mutant p53; tyrosinase; mucines, such as Muc-1, PSA, TSH, autoimmune antigens; immunotherapy drugs, such as AZT; and angiogenic and anti-angiogenic drugs, such as angiostatin, endostatin, basic fibroblast growth factor, and vascular endothelial growth factor, prostate specific antigen and thyroid stimulating hormone, GABA, acetyl choline, CD40 Ligand, the B7 Family of co-stimulatory factors, Anti-CTLA4, and BLYS.

Another type of agent includes hormones. Examples of such hormones include, but are not limited to, growth hormone, insulin, glucagon, parathyroid hormone, luteinizing hormone, follicle stimulating hormone, luteinizing hormone releasing hormone, estrogen, testosterone, dihydrotestoerone, estradiol, prosterol, progesterone, progestin, estrone, other sex hormones, and derivatives and analogs of hormones.

Yet another type of agent includes pharmaceuticals. Any type of pharmaceutical agent can be employed in the present invention. For example, antiinflammatory agents such as steroids and nonsteroidal antiinflammatory agents, soluble receptors, antibodies, antibiotics, analgesics, angiogenic and anti-angiogenic agents, and COX-2 inhibitors, can be employed in the present invention. Chemotherapeutic agents are of particular interest in the present invention. Nonlimiting examples of such agents include taxol, paclitaxel, taxanes, vinblastin, vincristine, doxorubicin, acyclovir, cisplatin methotrexate, mithramycin, melphalan and tacrine.

Immunotherapy agents are also of particular interest in the present invention. Nonlimiting examples of immunotherapy agents, include inflammatory agents, biological factors, immune regulatory proteins, and immunotherapy drugs, such as AZT and other derivatized or modified nucleotides. Small molecules can also be employed as agents in the present invention.

Another type of agent includes nucleic acid-based materials. Examples of such materials include, but are not limited to, nucleic acids, nucleotides, nucleotide analogs, DNA, RNA, tRNA, mRNA, sense nucleic acids, antisense nucleic acids, interference RNAs (RNAi), ribozymes, DNA, enzymes, protein/nucleic acid compositions, SNPs, oligonucleotides, vectors, viruses, plasmids, transposons, and other nucleic acid constructs knowvn to those skilled in the art.

Other agents that can be employed in the invention include, but are not limited to, ligands, cell surface receptors, antibodies, radioactive metals or molecules, detection agents, enzymes and enzyme co-factors.

Of particular interest are detection agents such as dyes or radioactive materials that can be used for visualizing or detecting the sequestered colloidal metal vectors. Fluorescent, chemiluminescent, heat sensitive, opaque, beads, magnetic and vibrational materials are also contemplated for use as detectable agents that are associated or bound to functionalized/reactive colloidal metals in the compositions of the present invention.

These agents may be employed separately, or in combinations. They may be employed in a free state or in complexes, such as in combination with a colloidal metal.

Targeting molecules are also components of compositions of the present invention. One or more targeting molecules may be directly or indirectly attached, bound or associated with the functionalized/reactive colloidal metal. These targeting molecules can be directed to specific cells or cell types, cells derived from a specific embryonic tissue, organs or tissues. Such targeting molecules include any molecules that are capable of selectively binding to specific cells or cell types. In general, such targeting molecules are one member of a binding pair and as such, selectively bind to the other member. Such selectivity may be achieved by binding to structures found naturally on cells, such as receptors found in cellular membranes, nuclear membranes or associated with DNA. The binding pair member may also be introduced synthetically on the cell, cell type, tissue or organ. Targeting molecules also include receptors or parts of receptors that may bind to molecules found in the cellular membranes or free of cellular membranes, ligands, antibodies, antibody fragments, enzymes, cofactors, substrates, and other binding pair members known to those skilled in the art. Targeting molecules may also be capable of binding to multiple types of binding partners. For example, the targeting molecule may bind to a class or family of receptors or other binding partners. The targeting molecule may also be an enzyme substrate or cofactor capable of binding several enzymes or types of enzymes.

Specific examples of targeting molecules include, but are not limited to, Interleukin-1 (“IL-1”), Interleukin-2 (“IL-2”), Interleukin-3 (“IL-3”), Interleukin-4 (“IL-4”), Interleukin-5 (“IL-5”), Interleukin-6 (“IL-6”), Interleukin-7 (“IL-7”), Interleukin-8 (“IL-8”), Interleukin-10 (“IL-10”), Interleukin-11 (“IL-11”), Interleukin-12 (“IL-12”), Interleukin-13 (“IL-13”), Interleukin-15 (“IL- 15”), Interleukin-16 (“IL-16”), Interleukin-17 (“IL-17”), Interleukin-18 (“IL-18”), CD40 Ligand, BLYS, B7, lipid A, phospholipase A2, endotoxins, staphylococcal enterotoxin B and other toxins, Type I Interferon, Type II Interferon, Tumor Necrosis Factor (“TNFα”), Transforming Growth Factor-α “TGF-α”), EGF, heat shock proteins, Lymphotoxin, Migration Inhibition Factor, Granulocyte-Macrophage Colony-Stimulating Factor (“CSF”), Monocyte-Macrophage CSF, Granulocyte CSF, vascular epithelial growth factor, Angiogenin, transforming growth factor-β (“TGF-β”), carbohydrate moieties of blood groups, Rh factors, fibroblast growth factor and other inflammatory and immune regulatory proteins, hormones, such as growth hormone, insulin, glucagon, parathyroid hormone, luteinizing hormone, follicle stimulating hormone, and luteinizing hormone releasing hormone, cell surface receptors, antibodies, nucleic acids, nucleotides, DNA, RNA, sense nucleic acids, antisense nucleic acids, cancer cell specific antigens, MART, MAGE, BAGE, and HSPs (Heat Shock Proteins), mutant p53; tyrosinase; antoimmune antigens; immunotherapy drugs, such AZT, and angiogenic and anti-angiogenic drugs, such as angiostatin, endostatin, vascular endothelial growth factor (VEGF), prostate specific antigen, thyroid stimulating hormone, receptor proteins, glucose, glycogen, phospholipids, and monoclonal and/or polyclonal antibodies, basic fibroblast growth factor, enzymes, cofactors and enzyme substrates.

Adjuvants useful in the invention include, but are not limited to, heat killed M. Butyricum and M. Tuberculosis. Nonlimiting examples of nucleotides are DNA, RNA, mRNA, sense, and antisense. Examples of immunogenic proteins include, but are not limited to, KLH (Keyhole Limpet Cyanin), thyroglobulin, CpG-motifs, toxins such as tetanus toxoid, sepharose, dextran and silica, BCG, and fusion proteins, which have adjuvant and antigen moieties encoded in the gene.

The integrating molecules used in the present invention can either be specific binding integrating molecules, such as members of a binding pair, or can be nonspecific binding integrating molecules that bind less specifically. The compositions of the present invention can comprise one or more integrating molecules. The integrating molecule as defined herein is a molecule that binds to a cell surface receptor and binds to the surface of the colloidal gold particles. This is in contrast to poly-1-lysine that is used as a reducing agent to form the functionalized/reactive colloidal gold particles. During the process of particle formation the “reducing” end of the derivatized poly-1-lysine becomes trapped in the core of the colloidal gold particle, leaving the poly-1-lysine swinging freely to serve as an attachment site for agents that may themselves serve as an integrating molecule or as a therapeutic agent.

Specific binding-integrating molecules comprise any members of binding pairs that can be used in the present invention. Such binding pairs are known to those skilled in the art and include, but are not limited to, antibody-antigen pairs, enzyme-substrate pairs, receptor-ligand pairs, and streptavidin-biotin. In addition to such known binding pairs, novel binding pairs may be specifically designed. A characteristic of binding pairs is the binding between the two members of the binding pair. Another desired characteristic of the binding partners is that one member of the pair is capable of binding or being bound to one or more of an agent or a targeting molecule, and the other member of the pair is capable of binding to the metal particle.

Proteins bind to the surface of the colloidal gold particles by one of three mechanisms. Two of these mechanisms, ionic and hydrophobic binding, are relatively weak interactions that oftentimes result in the generation of poor quality vectors. The third method involves formation of a dative (coordinate covalent) bond between free sulfhydryl/thiols of the biomolecule and the gold atoms present on the particle surface. Dative bonds are very stable, possessing the energy equivalence of a covalent bond, and are only disrupted by strong reducing agents such as dithiolthreitol or beta mercaptoethanol. Proteins that bind to the functionalized/reactive colloidal gold nanoparticles through dative bond formation are very stable and retain their biological activity.

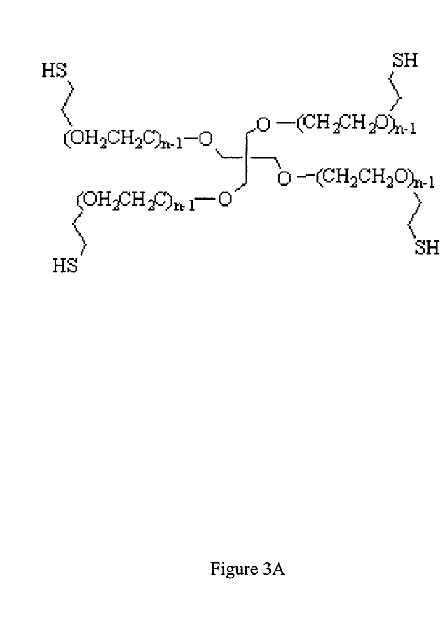

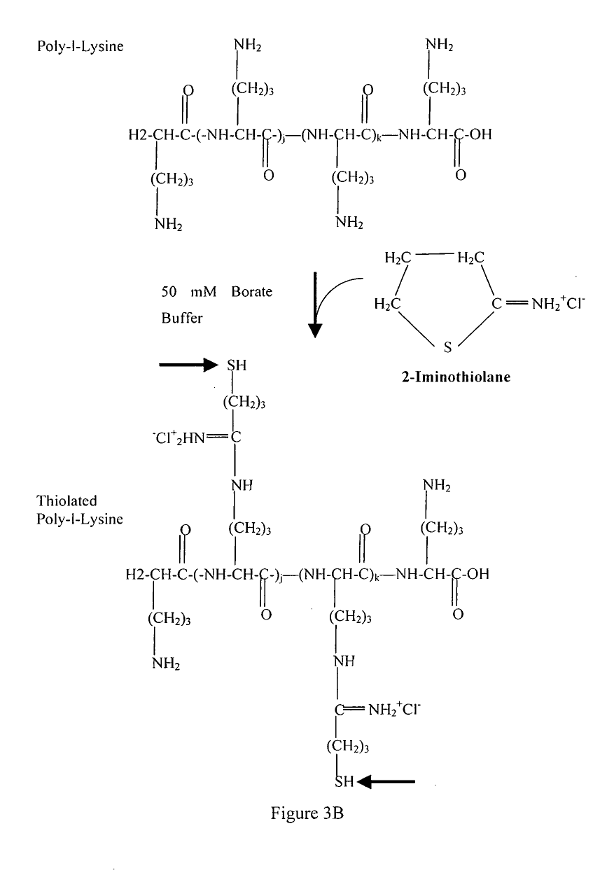

Another component of the compositions of the present invention comprises glycol compounds, preferably polyethylene glycol (PEG), more preferably derivatized PEG. A schematic of an example of this type of molecule consisting of 4 subunits of a 10 kD polymer of polyethylene glycol is shown in FIG. 3A. The present invention comprises compositions comprising derivatized PEG, wherein the PEG has a molecular weight range of 500 to 100,000 MW. In one embodiment of the present invention the molecular weight of derivatized PEG is between 5,000 to 80,000. In an alternative embodiment the molecular weight of derivatized PEG is approximately 5,000 to 60,000. In a further embodiment, the molecular weight of derivatized PEG is between 10,000 to 40,000. In a preferred embodiment the molecule weight of derivatized PEG is between 5,000 to 30,000. Derivatized PEG compounds are commercially available from sources such as Shearwater Corporation, Huntsville, Ala. or SunBio Inc. PEG compounds may be difunctional or monofunctional, such as methoxy-PEG (mPEG). The present invention contemplates use of any sized PEG with any derivative group, though preferred derivatized PEGs include mPEG-OPSS/2,000, mPEG-OPSS/5,000, mPEG-OPSS/10,000, mPEG-OPSS/12,000, mPEG-OPSS/20,000, mPEG-OP(SS)₂/2,000, mPEG-OP(SS)₂/3,400; mPEG-OP(SS)₂/8,000, mPEG-OP(SS)₂/10,000, thiol-PEG-thiol/2,000, mPEG-thiol 5,000, and mPEG thiol 10,000, mPEG thiol 12,000, mPEG thiol 20,000, 30,000, 40,000 (Sun-BIO Inc.). Activated derivatives of linear and branched PEGs are available in a variety of molecular weights. As used herein, the term “derivatized PEG(s)” or “PEG derivative(s)” means any polyethylene glycol molecule that has been altered with either addition of functional groups, chemical entities, or addition of other PEG groups to provide branches from a linear molecule. Such derivatized PEGs can be used for conjugation with biologically active compounds, preparation of polymer grafts, or other functions provided by the derivatizing molecule.

One type of PEG derivative is a polyethylene glycol molecule with primary amino groups at one or both of the termini. A preferred molecule is methoxy PEG with an amino group on one terminus. Another type of PEG derivative includes electrophilically activated PEG. These PEGs are used for attachment of PEG or methoxy PEG (mPEG), to proteins, liposomes, soluble and insoluble polymers and a variety of molecules. Electrophilically active PEG derivatives include succinimide of PEG propionic acid, succinimide of PEG butanoate acid, multiple PEGs attached to hydroxysuccinimide or aldehydes, mPEG double esters (mPEG-CM-HBA-NHS), mPEG benzotriazole carbonate, and mPEG propionaldehyde, mPEG acetaldehyde diethyl acetal.

A preferred type of derivatized PEG comprises thiol derivatized PEGs, or sulfhydryl-selective PEGs. Branched, forked or linear PEGs can be used as the PEG backbone that has a molecular weight range of 5,000 to 40,000 mw. Preferred thiol derivatized PEGs comprise PEG with maleimide functional group to which a thiol group can be conjugated. A preferred thiol-PEG is methoxy-PEG-maleimide, with PEG mw of 5,000 to 40,000.

Use of heterofunctional PEGs, as a derivatized PEG, is also contemplated by the present invention. Heterofunctional derivatives of PEG have the general structure X-PEG-Y. When the X and Y are functional groups that provide conjugation capabilities, many different entities can be bound on either or both termini of the PEG molecule. For example, vinylsulfone or maleimide can be X and an NHS ester can be Y. For detection methods, X and/or Y can be fluorescent molecules, radioactive molecules, luminescent molecules or other detectable labels. Heterofunctional PEG or monofunctional PEGs can be used to conjugate one member of a binding pair, such as PEG-biotin, PEG-Antibody, PEG-antigen, PEG-receptor, PEG-enzyme or PEG-enzyme substrate. PEG can also be conjugated to lipids such as PEG-phospholipids.

Another component of the compositions of the present invention comprises glycol compounds, preferably poly-1-lysine compositions, more preferably derivatized poly-1-lysine compositions. A schematic of an example of this molecule is shown in FIG. 3B. To make the poly-1-lysine, a suitable reducing agent such as 2-iminothiolane is used to thiolate the polymer on its multiple free amino groups.

As used herein, the term “derivatized poly-1-lysine(s)” or “poly-1-lysine derivative(s)” means any poly-1-lysine molecule that has been altered with either addition of functional groups, chemical entities, or addition of other poly-1-lysine groups to provide branches from a linear molecule. Such derivatized poly-1-lysine groups can be used for conjugation with biologically active compounds, preparation of polymer grafts, or other functions provided by the derivatizing molecule.

Thiolated alkanes are used to modify the surface of the colloidal metal nanoparticle. The alkane thiol acts as a bi-functional cross-linker between the gold particle and the agent since the thiol group serves to link the alkane thiol to the surface of the particle while the reactive groups acts as an acceptor molecule for the attachment of the agent.

One or more agents of the compositions of the present invention can be bound directly to the functionalized/reactive colloidal metal particles or can be bound indirectly to the colloidal metal through one or more integrating molecules. One method of preparing colloidal metal sols of the present invention uses the method described by Horisberger, (Biol. Cellulaire 36:253-258, 1979), which is incorporated by reference herein in its entirety. In embodiments where an integrating molecule is employed, the integrating molecule is bound to, admixed or associated with the metal sol. The agent may be bound to, admixed or associated with the integrating molecule prior to the binding, admixing or associating of the integrating molecule with the metal, or may be bound, admixed or associated after the binding of the integrating molecule to the metal.

General methods for binding agents to metal sols comprise the following steps. A solution of the agent is formed in a buffer or solvent, such as deionized water (diH₂O). The appropriate buffer or solvent will depend upon the agent to be bound. Determination of the appropriate buffer or solvent for a given agent is within the level of skill of the ordinary artisan. Determining the pH necessary to bind an optimum amount of agent to metal sol is known to those skilled in the art. The amount of agent bound can be determined by quantitative methods for determining proteins, therapeutic agents or detection agents, such as ELISA or spectrophotometric methods.

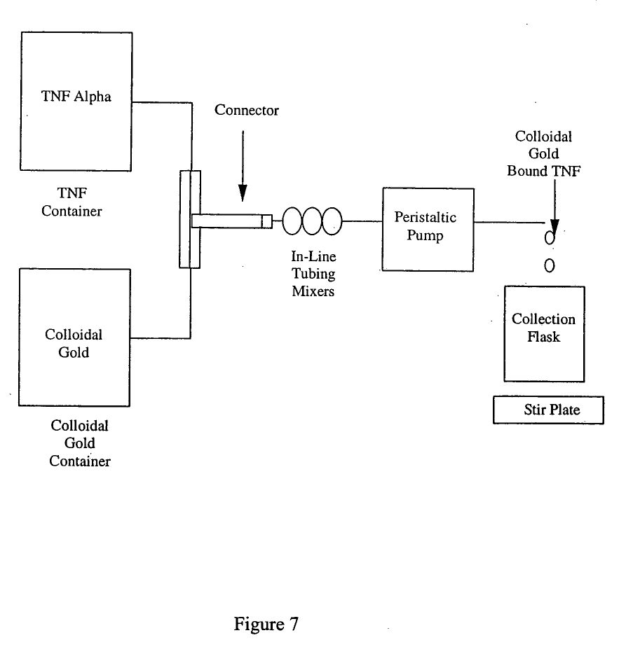

One method of binding an agent to a functionalized/reactive metal sol, such as thiolated metal sols, comprises the following steps, though for clarity purposes only, the methods disclosed refer to binding a single agent, TNF, to a thiolated metal sol, colloidal gold. An apparatus was used that allows interaction between the particles of the thiolated colloidal gold sol and TNF in a protein solution. A schematic representation of the apparatus is presented in FIG. 7. This apparatus maximizes the interaction of thiolated colloidal gold particles with the protein to be bound, TNF, by reducing the mixing chamber to a small volume. This apparatus enables the interaction of large volumes of gold sols with large volumes of TNF to occur in the small volume of a T connector. In contrast, adding a small volume of protein to a large volume of thiolated colloidal gold particles is not a preferred method to ensure uniform protein binding to the gold particles with respect to one another. Nor is the opposite method of adding small volumes of thiolated colloidal gold to a large volume of protein. Physically, the thiolated colloidal gold particles and the protein, TNF are forced into a T-connector by a single peristaltic pump that draws the thiolated colloidal gold particles and the TNF protein from two large reservoirs. To further ensure proper mixing, an in-line mixer is placed immediately down stream of the T-connector. The mixer vigorously mixes the thiolated colloidal gold particles with TNF, both of which are flowing through the connector at a preferable flow rate of approximately 1 L/min.

Prior to mixing with the agent, the pH of the gold sol is adjusted to pH 8-9 using 1 N NaOH. A preferred method for adjusting the pH of the gold sol uses 100 mM TRIS to adjust the pH of the thiolated colloidal gold sol to pH 6. Highly purified, lyophilized recombinant human TNF is reconstituted and diluted in 3 mM Tris and 0.25× solution (77.25 milli-osmol/kg) of normal phosphate buffered saline to a final concentration of TNF of 0.5 μg/ml. Before adding either the sol or TNF to their respective reservoirs, the tubing connecting the containers to the T-connector is clamped shut. Equal volumes of thiolated colloidal gold sol and the TNF solution are added to the appropriate reservoirs. In one embodiment, the concentration of agent in solution ranges from approximately 1 μg/ml to 50 μg/ml. (is this range too broad or acceptable. Preferred concentrations of agent in the solution range from approximately 0.01 to 15 μg/ml, and can be altered depending on the ratio of the agent to metal sol particles. Preferred concentrations of TNF in the solution range from 0.5 to 4 μg/ml and the most preferred concentration of TNF for the TNF-colloidal gold composition is 0.5 μg/ml. The present invention contemplates that one of ordinary skill in the art may achieve the appropriate or preferred concentration for each agent to be bound, admixed or associated with the thiolated metal sol.

Once the solutions are properly loaded into their respective reservoirs, the peristaltic pump is turned on, drawing the agent solution and the thiolated colloidal gold solution into the T-connector, through the in-line mixer, through the peristaltic pump and into a collection flask. The mixed solution is stirred in the collection flask for an additional hour of incubation.

In thiolated metal sol compositions that require additional PEG, whether derivatized or not, the methods for making such compositions comprise the following steps, though for clarity purposes only, the methods disclosed refer to adding PEG or PEG thiol to a thiolated metal sol composition. Any PEG, derivatized PEG composition or any sized PEG compositions or compositions comprising several different PEGs, can be made using the following steps. Following the 1-hour incubation taught above, a PEG or thiol derivatized polyethylene glycol (PEG) solution is added to the colloidal gold/TNF sol. The present invention contemplates use of any sized PEG with any derivative group, though preferred derivatized PEGs include mPEG-OPSS/5,000, thiol-PEG-thiol/3,400, mPEG-thiol 5000, and mPEG thiol 20,000 (Shearwater Polymers, Inc.). A preferred PEG is mPEG-thiol 5000 at a concentration of 150 μg/ml in water, pH 5-8. Thus, a 10% v/v of the PEG solution is added to the colloidal gold-TNF solution. The gold/TNF/PEG solution is incubated for an additional hour.

In an alternative embodiment, the TNF and PEG-thiol moiety simultaneously bind to the thiolated colloidal gold nanoparticle. In this method the pH of the colloidal gold nanoparticles is adjusted to 6.0 using 100 mM TRIS Base. Similarly the pH of water is adjusted to 6.0 using the 100 mM TRIS solution. Into the latter solution TNF and PEG-thiol (20,000) are diluted to a final concentration of 5 and 15 μg/ml, respectively. Both the thiolated colloidal gold nanoparticles and TNF/PEG-thiol solutions are loaded into their respective reservoirs and bound through the T-connector and in-line mixer using a peristaltic pump to draw each solution through the T-connector. After binding for 15 minutes Human Serum Albumin (200 μg/ml in H₂O) is added to the thiolated colloidal gold/TNF/PEG-thiol solution and incubated for an additional 15 minutes.

The colloidal gold/TNF/PEG solution is subsequently ultrafiltered through a 50K MWCO diafiltration cartridge. The 50K retentate and permeate are measured for TNF concentration by ELISA to determine the amount of TNF bound to the gold particles.

After diafiltration, cryoprotectants, including, but not limited to, compositions of mannitol, 20 mg/ml; and/or human serum albumin, 5 mg/ml, are added and the samples frozen at −80° C. The samples are lyophilized to dryness and sealed under a vacuum, subsequently reconstituted and may be analyzed for the amount of free and colloidal gold bound TNF present in the reconstituted samples or utilized directly.

The compositions of the present invention can be administered to in vitro and in vivo systems. In vivo administration may include direct application to the target cells or such routes of administration, including but not limited to formulations suitable for oral, rectal, transdermal, ophthalmic, (including intravitreal or intracameral) nasal, topical (including buccal and sublingual), vaginal or parenteral (including subcutaneous, intramuscular, intravenous, intradermal, intratracheal, and epidural) administration. A preferred method comprising administering, via oral or injection routes, an effective amount of a composition comprising vectors of the present invention.

The formulations may conveniently be presented in unit dosage form and may be prepared by conventional pharmaceutical techniques. Pharmaceutical formulation compositions are made by bringing into association the metal sol vectors and the pharmaceutical carrier(s) or excipient(s). In general, the formulations are prepared by uniformly and intimately bringing into association the compositions with liquid carriers or finely divided solid carriers or both, and then, if necessary, shaping the product.

Preferred methods of use of the compositions of the present invention comprise targeting the vectors to tumors. Preferred vector compositions comprise functionalized/reactive metal particles, agents and PEG, derivatized PEG, poly-1-lysine, or derivatized poly-1-lysine compositions for delivery to a tumor for therapeutic effects on the tumor or organism or detection of tumors. Such vector compositions may further comprise targeting and/or integrating molecules. Still other preferred vector compositions comprise functionalized/reactive metal particles, radioactive or cytotoxic agents and PEG, derivatized PEG, poly-1-lysine, or derivatized poly-1-lysine compositions for delivery of radiation therapies to tumors. Historically, radioactive colloidal gold was used as a cancer therapy, principally for the treatment of liver cancer due to the anticipated uptake of colloidal gold by the liver cells. Preferred compositions of the instant invention comprising derivatized PEG, preferably PEG thiol (PEG(SH)ₙ, in combination with radioactive functionalized colloidal metal particles are used to treat or identify tumors. Alternatively, compositions comprising derivatized poly-1-lysine, preferably poly-1-lysine thiol (PLL(SH)ₙ, in combination with radioactive colloidal metal particles may also be used to treat or identify tumors. Alternatively, a vector composition comprising a radioactive moiety coupled to a protein that is bound to colloidal metal, and further comprising derivatized PEG, preferably PEG-thiol, or derivatized poly-1-lysine, preferably poly-1-lysine thiol, forming a radioactive vector, is used to treat tumors. The radioactive vector composition of the present invention is injected intravenously and traffics to the tumor and is not significantly taken up by the liver. In both compositions, it is believed that the ability of the PEG thiol or the poly-1-lysine thiol to concentrate the radioactive therapy in the tumor increases treatment efficacy while reducing treatment side effects. It is contemplated that the compositions of the instant invention are particularly suited to the treatment, detection and imaging of solid tumors. In a preferred embodiment the compositions of the instant invention are directed to the treatment of solid tumors.

The present invention comprises compositions for use in methods for delivery of exogenous nucleic acids or genetic material into cells. The exogenous genetic material may be targeted to specific cells using targeting molecules that are capable of recognizing the specific cells or specifically targeted to tumors using compositions comprising PEG, derivatized PEG, poly-1-lysine or derivatized poly-1-lysine. For example, the targeting molecule is a binding partner for a specific receptor on the cells, and after binding, the entire composition may be internalized within the cells. The binding of the vector composition may activate cellular mechanisms that alter the state of the cell, such as activation of secondary messenger molecules within the cell. Thus, in a mixture of different cell types, the exogenous nucleic acids are delivered to cells having the selected receptor and cells lacking the receptor are unaffected.

The present invention comprises compositions and methods for the transfection of specific cells, in vitro or in vivo, for insertion or application of agents. One embodiment of such a composition comprises nucleic acid bound to polycations that are bound to colloidal metals. A preferred embodiment of the present invention comprises colloidal gold as a platform that is capable of binding targeting molecules and nucleic acid agents to create a targeted gene delivery vector that employs receptor-mediated endocytosis of cells to achieve transfection. In a more preferred embodiment, the targeting molecule is a cytokine and the agent is genetic material such as DNA or RNA. This embodiment may also comprise integrating molecules to which the genetic material is bound or associated.

In the present invention, the methods comprise the preparation of gene delivery vectors and delivery of the targeted gene delivery vector to the cells for transfection or therapeutic effects. It is contemplated in the present invention that the nucleic acids of the compositions may be internalized and used as detection agents or for genetic therapeutic effects, or the nucleic acids can be translated and expressed by the cell. The expression products can be any known to those skilled in the art and includes but is not limited to functioning proteins, production of cellular products, enzymatic activity, export of cellular products, production of cellular membrane components, or nuclear components. The methods of delivery to the targeted cells may be such methods as those used for in vitro techniques such as with cellular cultures, or those used for in vivo administration. In vivo administration may include direct application to the cells or such routes of administration as used for humans, animals or other organism, preferably intravenous or oral administration. The present invention also contemplates cells that have been altered by the compositions of the present invention and the administration of such cells to other cells, tissues or organisms, by in vitro or in vivo methods.

The present invention comprises compositions and methods for enhancing an immune response and increasing vaccine efficacy through the simultaneous or sequential targeting of specific immune cells using compositions directed to specific immune components. The compositions can also be used in methods for imaging or detecting immune cells. These methods comprise vector compositions comprising a functionalized/reactive colloidal metal particle and at least one agent capable of affecting the immune system. In one embodiment the compositions comprise functionalized/reactive colloidal metals associated with at least one of the following components, targeting molecules, agents, integrating molecules, one or more types of PEG, derivatized PEG, poly-1-lysine or derivatized poly-1-lysine. The vector compositions may also comprise specific immune components, such as cells including, but not limited to, antigen presenting cells (APCs), such as macrophages and dendritic cells, and lymphocytes, such as B cells and T cells, which have been or are individually effected by one or more component-specific immunostimulating agents.

Examples of component-specific immunostimulating molecules include, but are not limited to, Interleukin-1 (“IL-1”), Interleukin-2 (“IL-2”), Interleukin-3 (“IL-3”), Interleukin-4 (“IL-4”), Interleukin-5 (“IL-5”), Interleukin-6 (“IL-6”), Interleukin-7 (“IL-7”), Interleukin-8 (“IL-8”), Interleukin-10 (“IL-10”), Interleukin-11(“IL-11”), Interleukin-12 (“IL-12”), Interleukin-13 (“IL-13”), lipid A, phospholipase A2, endotoxins, staphylococcal enterotoxin B and other toxins, Type I Interferon, Type II Interferon, Tumor Necrosis Factor (“TNF-α”), Transforming Growth Factor-β (“TGF-β”), Lymphotoxin, Migration Inhibition Factor, Granulocyte-Macrophage Colony-Stimulating Factor (“CSF”), Monocyte-Macrophage CSF, Granulocyte CSF, vascular epithelial growth factor, Angiogenin, transforming growth factor (“TGF-α”), heat shock proteins, carbohydrate moieties of blood groups, Rh factors, fibroblast growth factor, and other inflammatory and immune regulatory proteins, nucleotides, DNA, RNA, mRNA, sense, antisense, cancer cell specific antigens; such as MART, MAGE, BAGE; flt3 ligand/receptor system; B7 family of molecules and receptors; CD 40 ligand/receptor; and immunotherapy drugs, such as AZT; and angiogenic and anti-angiogenic drugs, such as angiostatin, endostatin, and basic fibroblast growth factor, or vascular endothelial growth factor (“VEGF”).

An especially preferred embodiment provides methods for activation of the immune response using vector compositions comprising a functionalized/reactive colloidal metal particle and at least one agent, wherein the agents comprise a specific antigen in combination with a component-specific immunostimulating agent. Such methods are effective and can be used in vitro or in vivo. As used herein, component-specific immunostimulating agent means an agent that is specific for a component of the immune system, such as a B or T cell, and that is capable of affecting that component, so that the component has an activity in the immune response. The component-specific immunostimulating agent may be capable of affecting several different components of the immune system, and this capability may be employed in the methods and compositions of the present invention. The agent may be naturally occurring or can be generated or modified through molecular biological techniques or protein receptor manipulations.

The activation of the component in the immune response may result in a stimulation or suppression of other components of the immune response, leading to an overall stimulation or suppression of the immune response. For ease of expression, stimulation of immune components is described herein, but it is understood that all responses of immune components are contemplated by the term stimulation, including but not limited to stimulation, suppression, rejection and feedback activities.

The immune component that is affected may have multiple activities, leading to both suppression and stimulation or initiation or suppression of feedback mechanisms. The present invention is not to be limited by the examples of immunological responses detailed herein, but contemplates component-specific effects in all aspects of the immune system.

The activation of each of the components of the immune system may be simultaneous, sequential, or any combination thereof. In one embodiment of a method of the present invention, multiple component-specific immunostimulating agents are administered simultaneously. In this method, the immune system is simultaneously stimulated with multiple separate preparations, each containing a vector composition comprising a component-specific immunostimulating agent. Preferably, the vector composition comprises the component-specific immunostimulating agent associated with the functionalized/reactive colloidal metal. More preferably, the composition comprises the component-specific immunostimulating agent associated with the functionalized/reactive colloidal metal of one sized particle or of different sized particles and an antigen. Most preferably, the composition comprises the component-specific immunostimulating agent associated with the functionalized/reactive colloidal metal of one sized particle or of differently sized particles, antigen and PEG, derivatized PEG, poly-1-lysine or derivatized poly-amino acid such as poly-1-lysine.

Component-specific immunostimulating agents provides a specific stimulatory, up regulation, effect on individual immune components. For example, Interleukin-1β (IL-1β) specifically stimulates macrophages, while TNF-α (Tumor Necrosis Factor alpha) and Flt-3 ligand specifically stimulate dendritic cells. Heat killed Mycobacterium butyricum and Interleukin-6 (IL-6) are specific stimulators of B cells, and Interleukin-2 (IL-2) is a specific stimulator of T cells. Vector compositions comprising such component-specific immunostimulating agents provide for specific activation of macrophages, dendritic cells, B cells and T cells, respectively. For example, macrophages are activated when a vector composition comprising the component-specific immunostimulating agent IL-1β is administered. A preferred composition is IL-1β in association with functionalized/reactive colloidal metal, and a most preferred composition is IL-1β in association with functionalized/reactive colloidal metal and an antigen to provide a specific macrophage response to that antigen. Vector compositions can further comprise targeting molecules, integrating molecules, PEGs, derivatized PEGs, poly-1-lysine or derivatized poly-amino acid such as poly-1-lysine.

Many elements of the immune response may be necessary for an effective immune response to an antigen. An embodiment of a method of simultaneous stimulation is to administer four separate preparations of compositions of component-specific immunostimulating agents comprising 1) IL-1β for macrophages, 2) TNF-α and Flt-3 ligand for dendritic cells, 3) IL-6 for B cells, and 4) IL-2 for T cells. Each component-specific immunostimulating agent vector composition may be administered by any routes known to those skilled in the art, and all may use the same route or different routes, depending on the immune response desired.

In another embodiment of the methods and compositions of the present invention, the individual immune components are activated sequentially. For example, this sequential activation can be divided into two phases, a primer phase and an immunization phase. The primer phase comprises stimulating APCs, preferably macrophages and dendritic cells, while the immunization phase comprises stimulating lymphocytes, preferably B cells and T cells. Within each of the two phases, activation of the individual immune components may be simultaneous or sequential. For sequential activation, a preferred method of activation is administration of vector compositions that cause activation of macrophages followed by dendritic cells, followed by B cells, followed by T cells. A most preferred method is a combined sequential activation comprising the administration of vector compositions which cause simultaneous activation of the macrophages and dendritic cells, followed by the simultaneous activation of B cells and T cells. This is an example of methods and compositions of multiple component-specific immunostimulating agents to initiate several pathways of the immune system.

The methods and compositions of the present invention can be used to enhance the effectiveness of any type of vaccine. The present methods enhance vaccine effectiveness by targeting specific immune components for activation. Vector compositions comprising at least component-specific immunostimulating agents in association with functionalized/reactive colloidal metal and antigen are used for increasing the contact between antigen and the specific immune component, such as macrophages, B or T cells. Examples of diseases for which vaccines are currently available include, but are not limited to, cholera, diphtheria, Haemophilus, hepatitis A, hepatitis B, influenza, measles, meningitis, mumps, pertussis, small pox, pneumococcal pneumonia, polio, rabies, rubella, tetanus, tuberculosis, typhoid, Varicella-zoster, whooping cough, and yellow fever.

The combination of routes of administration and the vector compositions for delivering the antigen to the immune system is used to create the desired immune response. The present invention also comprises methods and compositions comprising various compositions of packaging systems, such as liposomes, microcapsules, or microspheres, that can provide long-term release of immune stimulating vector compositions. These packaging systems act as internal depots for holding antigen and slowly releasing antigen for immune system activation. For example, a liposome may be filled with a vector composition comprising the agents of an antigen and component-specific immunostimulating agent, bound to or associated with a functionalized/reactive colloidal metal. Additional combinations are functionalized/reactive colloidal gold particles studded with agents such as viral particles, which are the active vaccine candidate or are packaged to contain DNA for a putative vaccine. The vector may also comprise one or more targeting molecules, such as a cytokine, integrating molecules, PEG derivatives or poly-1-lysine derivatives, and the vector is then used to target the virus to specific cells. Furthermore, one could use a fusion protein vaccine, which targets two or more potential vaccine candidates, and provide a vector composition vaccine that provides protection against two or more infectious microorganisms. The compositions may also include immunogens, which have been chemically modified by the addition of polyethylene glycol, which may release the material slowly.

The compositions comprising a functionalized/reactive metal particle and the agent comprising one or more antigens and one or more component-specific immunostimulating agents, and one or more of integrating and targeting molecules and PEG, derivatives of PEG, poly-1-lysine or derivatives of poly-1-lysine, may be packaged in a liposome or a biodegradable polymer. The vector composition is slowly released from the liposome or biodegradable polymer and is recognized by the immune system as foreign and the specific component to which the component-specific immunostimulating agent is directed activates or suppresses the immune system. For example, the cascade of the immune response is activated more quickly by the presence of the component-specific immunostimulating agent and the immune response is generated more quickly and more specifically.

Other methods and compositions contemplated in the present invention include using compositions of functionalized/reactive metal particles and agents comprising antigens and component-specific immunostimulating agents, which may also comprise integrating and targeting molecules, in which the functionalized/reactive colloidal metal particles have different sizes. The compositions may further comprise PEG, derivatives of PEG, poly-1-lysine or derivatives of poly-1-lysine. Sequential administration of component-specific immunostimulating agents may be accomplished in a one dose administration by use of differently sized functionalized/reactive colloidal metal particles. One dose would include multiple independent component-specific immunostimulating agents an antigen and the combination could be associated with a differently sized or the same sized functionalized/reactive colloidal metal particle. Thus, simultaneous administration would provide sequential activation of the immune components to yield a more effective vaccine and more protection for the population. Other types of such single-dose administration with sequential activation could be provided by combinations of differently sized or same sized functionalized/reactive colloidal metal vector compositions and liposomes or biodegradable polymers, or liposomes or biodegradable polymers filled with differently sized or same-sized functionalized/reactive colloidal metal vector compositions.

Use of such vaccination systems as described above are important in providing vaccines that can be administered in one dose. One dose administration is important in treating animal populations such as livestock or wild populations of animals. One dose administration is vital in treatment of populations for whom healthcare is rarely accessible such as the poor, homeless, rural residents or persons in developing countries that have inadequate health care. Many persons, in all countries, do not have access to preventive types of health care, such as vaccination. The reemergence of infectious diseases, such as tuberculosis, has increased the demand for vaccines that can be given once and still provide long-lasting, effective protection. The compositions and methods of the present invention provide such effective protection.

Many diseases, in addition to cancer, are mediated by the immune system and the present invention comprises methods of treatment of such diseases by the administration of an effective amount of a composition comprising a functionalized/reactive colloidal metal vector that is capable of stimulating the immune system and it components. The methods and compositions of the present invention can also be used to treat diseases in which an immune response occurs, by stimulating or suppressing components that are a part of the immune response. Examples of such diseases include, but are not limited to, Addison's disease, Crohn's disease, inflammatory bowel disease, adult respiratory distress syndrome, hand foot and mouth disease, allergies, anaphylaxis, Bruton's syndrome, cancer, including solid and blood borne tumors, eczema, Hashimoto's thyroiditis, polymyositis, dermatomyositis, type 1 diabetes mellitus, acquired immune deficiency syndrome, transplant rejection, such as kidney, heart, pancreas, lung, bone, and liver transplants, Graves' disease, polyendocrine autoimmune disease, hepatitis, microscopic polyarteritis, polyarteritis nodosa, pemphigus, primary biliary cirrhosis, pernicious anemia, coeliac disease, antibody-mediated nephritis, glomerulonephritis, rheumatic diseases, systemic lupus erthematosus, rheumatoid arthritis, seronegative spondylarthritides, rhinitis, sjogren's syndrome, systemic sclerosis, sclerosing cholangitis, Wegener's granulomatosis, dermatitis herpetiformis, psoriasis, vitiligo, multiple sclerosis, encephalomyelitis, Guillain-Barre syndrome, myasthenia gravis, Lambert-Eaton syndrome, sclera, episclera, uveitis, chronic mucocutaneous candidiasis, urticaria, transient hypogammaglobulinemia of infancy, myeloma, X-linked hyper IgM syndrome, Wiskott-Aldrich syndrome, ataxia telangiectasia, autoimmune hemolytic anemia, autoimmune thrombocytopenia, autoimmune neutropenia, Waldenstrom's macroglobulinemia, amyloidosis, chronic lymphocytic leukemia, and non-Hodgkin's lymphoma.

Preferred embodiments of the vector compositions comprise agents comprising component-specific immunostimulating agents in association with functionalized/reactive colloidal metals. More preferred embodiments comprise compositions comprising agents comprising one or more antigens and component-specific immunostimulating agents in association with functionalized/reactive colloidal metals and at least one of the following, PEG or derivatives of PEG, poly-1-lysine or derivatives of poly-1-lysine, integrating molecules and targeting molecules for specifically targeting the effect of the component-specific immunostimulating agents, including, but not limited to, antigens, receptor molecules, nucleic acids, pharmaceuticals, chemotherapy agents, and carriers. The compositions of the present invention may be delivered to the immune components in any manner. In one embodiment, the agents, comprising an antigen and a component-specific immunostimulating agent, are bound to a functionalized/reactive colloidal metal in such a manner that a functionalized/reactive colloidal metal particle is associated with both the antigen and the immunostimulating agent.

The present invention includes presentation of agents such as antigen and component-specific immunostimulating agents in a variety of different delivery platforms or carrier combinations. For example, a preferred embodiment includes administration of a vector composition comprising a functionalized/reactive metal colloid particle bound to agents such as an antigen and component-specific immunostimulating agents in a liposome or biodegradable polymer carrier. Additional combinations are functionalized/reactive colloidal gold particles associated with agents such as viral particles which are the vaccine antigen or which are viable viral particles containing nucleic acids that produce antigens for a vaccine. The vector compositions may also comprise targeting molecules such as a cytokine or a selected binding pair member which is used to target the virus to specific cells, and further comprises other elements taught herein such as integrating molecules or PEG, PEG derivatives, poly-1-lysine or poly-amino acid derivatives such as poly-1-lysine. Such embodiments provide for a vaccine preparation that slowly releases antigen to the immune system for a prolonged response. This type of vaccine is especially beneficial for one-time administration of vaccines. All types of carriers, including but not limited to liposomes and microcapsules are contemplated in the present invention.

Toxicity Reduction and Vaccine Administration

The present invention comprises compositions and methods for administering factors that, when the factors are present in higher than normal concentrations, are toxic to a human or animal. Generally, the compositions according to the present invention comprise a vector composition that is an admixture of a functionalized/reactive colloidal metal in combination with an agent which is toxic to a human or animal when the agent is found in higher than normal concentration, or is in an unshielded form that allows for greater activity than in a shielded form, or is found in a site where it is not normally found. When the vector composition is administered to a human or animal, the agent is less harmful or less toxic or non-toxic to the human or animal than when the agent is provided alone without the functionalized/reactive colloidal metal vector composition. The compositions optionally include a pharmaceutically-acceptable carrier, such as an aqueous solution, or excipients, buffers, antigen stabilizers, or sterilized carriers. Also, oils, such as paraffin oil, may optionally be included in the composition. The vector compositions may further comprise PEG, derivatives of PEG, poly-1-lysine or derivatives of poly-1-lysine.

The compositions of the present invention can be used to vaccinate a human or animal against agents that are toxic when injected. In addition, the present invention can be used to treat certain diseases with cytokines or growth factors by administering the compositions comprising agents such as cytokines or growth factors. By admixing the agents with the functionalized/reactive colloidal metal before administering the agents to the human or animal, the toxicity of the agent is reduced or eliminated thereby allowing the factor to exert its therapeutic effect. The combination of a functionalized/reactive colloidal metal with such agents in a vector composition reduces toxicity while maintaining or increasing the therapeutic results thereby improving the efficacy as higher concentrations of agents may be administered, or by allowing the use of combinations of different agents. The use of functionalized/reactive colloidal metals in combination with agents in vector compositions therefore allows the use of higher than normal concentrations of agents or administration of agents that normally are unusable due to their toxicity, to be administered to humans or animals. Preferably, the vector compositions further comprise one or more types or sizes of PEG, derivatives of PEG, poly-1-lysine or derivatives of poly-amino acids such as poly-1-lysine.

One embodiment of the present invention comprises methods for using a vector composition comprising an agent associated with the functionalized/reactive colloidal metal as a vaccine preparation. Among the many advantages of such a vaccine is the reduction of toxicity of normally toxic agents. The vector compositions used as a vaccine against agents may be prepared by any method. For example, the vector composition of an admixture of agents and functionalized/reactive colloidal metal is preferably injected into an appropriate animal. Because the agent is not toxic when administered according to the present invention, the optimal quantity of the agent, which can function as an antigen, can be administered to the animal. The vector compositions according to the present invention may be administered in a single dose or may be administered in multiple doses, spaced over a suitable time scale. Multiple doses are useful in developing a secondary immunization response. For example, antibody titers have been maintained by administering boosters once a month.

The present invention is advantageous for vaccine preparations due to the high amount of agent delivered in the lyophilized compositions. The lyophilized compositions have a longer shelf life than non-lyophilized compositions and can be more easily transported than non-lyophilized compositions.

The vaccine compositions may further comprise a pharmaceutically acceptable adjuvant, including, but not limited to Freund's complete adjuvant, Freund's incomplete adjuvant, lipopolysaccharide, monophosphoryl lipid A, muramyl dipeptide, liposomes containing lipid A, alum, muramyl tripeptidephosphatidylethanoloamine, keyhole limpet hemocyanin. A preferred adjuvant for animals is Freund's incomplete adjuvant and Alum for humans, which preferably is diluted 1:1 with the compositions comprising a functionalized/reactive colloidal metal and an active agent.

A preferred method of use of the compositions of the present invention comprises administering to a human or animal an effective amount of a vector composition comprising a functionalized/reactive colloidal metal admixed with at least one agent, wherein the composition when administered to a human or animal, is less or non-toxic, or has fewer or less severe side effects when compared to administration of the agent alone or in compositions without functionalized/reactive colloidal metals. The vector compositions according to the present invention can be administered as a vaccine against a normally toxic substance or can be a therapeutic agent wherein the toxicity of the normally toxic agent is reduced thereby allowing the administration of higher quantities of the agent over longer periods of time.

In practicing these embodiments, the route by which the composition is administered is not considered critical. The routes that the composition may be administered according to this invention include known routes of administration, including, but are not limited to, subcutaneous, intramuscular, intraperitoneal, oral, and intravenous routes. A preferred route of administration is intravenous. Another preferred route of administration is intramuscular.

For example, it is known that Interleukin-2 (IL-2) displays significant therapeutic results in the treatment of renal cancer. However, the toxic side effects of administration of IL-2 result in the death of a significant number of the patients.

The present invention comprises methods for treating diseases by administering vector compositions comprising one or more agents and a functionalized/reactive colloidal metal. The vector compositions may further comprise PEG, derivatives of PEG, poly-1-lysine or derivatives of poly-1-lysine. It is contemplated by the instant invention that the agent may be optionally released from the functionalized/reactive colloidal metal. Though not wishing to be bound by any theory, it is thought that the release is not simply a function of the circulation time, but is controlled by equilibrium kinetics and the presence of other ions and reducing agents in the body. In this regard, the instant invention contemplates the use of a trigger to initiate release of the agent from the functionalized/reactive colloidal metal particle when such action is required. In one embodiment, an effective amount of areducing agent may be administered to a site, cell or location, following the administration of the functionalized/reactive colloidal metal vector composition. In another embodiment, the release of an agent, for example an active drug, from the functionalized/reactive colloidal gold particle may occur by the addition of agents, such as molecules or compounds capable of reducing the thiol bond that binds the agent to the functionalized/reactive colloidal metal particle.

It is theorized that due to the continuous in vivo dilution of the compositions by blood and extracellular fluids, it is possible to achieve the same therapeutic effect by administering a lower dose of an agent to a patient than can be administered by previously known methods.

Thus, the skilled artisan could control the amount of agent delivered by varying the amount of agent initially bound to the colloidal metal and the amount of reducing agent administered to reduce the thiol bond binding the agent to the functionalized/reactive colloidal metal particle.

The compositions of the present invention are useful for the treatment of a number of diseases including, but not limited to, cancer, both solid tumors as well as blood-borne cancers, such as leukemia; autoimmune diseases, such as rheumatoid arthritis; hormone deficiency diseases, such as osteoporosis; hormone abnormalities due to hypersecretion, such as acromegaly; infectious diseases, such as septic shock; genetic diseases, such as enzyme deficiency diseases (e.g., inability to metabolize phenylalanine resulting in phenylketanuria); and immune deficiency diseases, such as AIDS.

Methods of the present invention comprise administration of the vector compositions in addition to currently used therapeutic treatment regimens. Preferred methods comprise administering vector compositions concurrently with administration of therapeutic agents for treatment of chronic and acute diseases, and particularly cancer treatment. For example, a vector composition comprising the agent, TNF, is administered prior to, during or after chemotherapeutic treatments with known anti-cancer agents such as antiangiogenic proteins such as endostatin and angiostatin, thalidomide, taxol, melphalan, paclitaxel, taxanes, vinblastin, vincristine, doxorubicin, acyclovir, cisplatin and tacrine. All currently known cancer treatment methods are contemplated in the methods of the present invention and the vector compositions may be administered at different times in the treatment schedule as necessary for effective treatment of the cancer.

A preferred method comprises treatment of drug-resistant tumors, cancer or neoplasms. These tumors are resistant to known anti-cancer drugs and therapeutics and even with increasing dosages of such agents, there is little or no effect on the size or growth of the tumor. Known in cancer treatment is the observation that exposure of such drug resistant tumor cells to TNF resensitizes these cells to the anti-cancer effect of these chemotherapeutics. Evidence has been published that indicates that TNF synergizes with topoisomerase II-targeted intercalative drugs such as doxorubicin to restore doxorubicin tumor cell death. Also interferon (IFN) is known to synergize with 5-fluorouracil to increase the chemotherapeutic activity of 5-fluorouracil. The present invention can be used to treat such drug-resistant tumors. A preferred method comprises administration of vector compositions comprising TNF and functionalized/reactive colloidal gold. With the pretreatment of a patient with a subclinical dose of TNF-cAu-PT, the tumor sequesters the TNF vector, sensitizing the cells to subsequent systemic chemotherapy. Such chemotherapies include, but are not limited to doxorubicin, other intercalative chemotherapies, taxol, 5-fluorouracil, mitaxantrone, VM-16, etoposide, VM-26, teniposide, and other non-intercalative chemotherapies. Alternatively, another preferred method comprises administration of the above vector composition comprising TNF and at least one other agent effective for the treatment of cancer. For example, a PT-cAU_{(TNF)}doxorubicin vector is administered to patients who have drug resistant tumors or cancer. The amount administered is dependent on the tumor or tumors to be treated and the condition of the patient. The vector composition allows for greater amounts of the chemotherapeutic agents to be administered and the vector also relieves the drug-resistant characteristic of the tumor.

This invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLES

### Example 1

### A Method of Preparation of Colloidal Gold Sols

Colloidal gold is produced by the reduction of chloroauric acid (Au⁺³; HAuCl₄), to neutral gold (Au⁰) by agents such as sodium citrate. The method described by Horisberger, (1979) was adapted to produce 34 nm colloidal gold particles. This method provided a simple and scalable procedure for the production of colloidal gold. Briefly, a 4% gold chloride solution (23.03% stock; dmc², South Plainfield, N.J.) and a 1% sodium citrate solution (wt/wt; J.T. Baker Company; Paris, Ky.) were made in de-ionized H₂O (DIH₂O). 3.75 ml of the gold chloride solution was added to 1.5 L of DIH₂O. The solution was vigorously stirred and brought to a rolling boil under reflux. The formation of 34 nm colloidal gold particles was initiated by the addition of 60 ml of sodium citrate. The solution was continuously boiled and stirred during the entire process of particle formation and growth as described below.

The addition of sodium citrate to the gold chloride initiated a series of reduction reactions characterized by changes in the color of the initial gold chloride solution. With the addition of the sodium citrate the color of the gold chloride solution changed from a golden yellow to clear, and then an intermediate color of black/brown. The completion of the reaction was signaled by a final color change in the sol from brown/black to cherry red. After the final color change the solution was continuously stirred and boiled under reflux for an additional 45 minutes. Subsequently, the sol was cooled to room temperature and filter through a 0.22 u cellulose nitrate filter and stored at RT until use.

The formation of colloidal gold particles occurs in three stages: nucleation and particle growth and coagulation. Particle nucleation was initiated by the reduction of Au⁺³ to Au⁰ by sodium citrate. This step is marked by a color change of the gold chloride solution from bright yellow to black. The continuous layering of free Au⁺³ onto the Au⁰ nuclei drives the second stage, particle growth. Particle size is inversely related to the amount of citrate added to the gold chloride solution: increasing the amount of sodium citrate to a fixed amount of gold chloride results in the formation of smaller particles, while reducing the amount of citrate added to the gold solution results in the formation of relatively larger particles.

Similar to the nucleation reaction, colloidal gold particle formation is also correlated with a change in the solution's color. However, unlike the initial reaction, this second color change is directly related to particle size. When small particles (i.e., 12-17 nm) are made the sol is orange to red in color; when medium sized particles (i.e., 20-40 nm) are made the sol appears red to burgundy in color and when large particles (i.e., 64-97 nm) are made the sols appear brown in color. Critical to both particle nucleation and growth was the vigorous stirring of the reactants. Inadequate stirring at any step during the process resulted in the formation of heterogeneous particles with larger than predicted diameters.

TEM (transmission electron microscopy) and dual angle light scattering interrogation of the colloidal gold preparations revealed that the size of the particles in the colloidal gold preparations were very close to their theoretical size of 34 nm. The particles were homogenous in size with a mean particle diameter of 34-36 nm and a polydispersity measure averaging 0.11. In this state the colloidal gold particles stayed in suspension by their mutual electrostatic repulsion due to the negative charge present on each particle's surface. Exposing these naked particles to salt solutions (i.e., NaCl at a 1% v/v final concentration) caused them to aggregate and ultimately precipitate out of solution. This process was blocked or inhibited by binding proteins (e.g., TNF) or other agents to the particles' surface.

### Example 2

### Manufacture of Derivatized Poly-L-Lysine

Derivatized poly-1-lysine was generated using the thiolating agent, 2-iminothiolane to thiolate the free amino group present on the lysine residues of a poly-1-lysine (PLL) polymer backbone. To generate this reagent 94 mg of poly-1-lysine (MW=14600) was diluted in 10 ml of a 50 mM sodium borate buffer. Subsequently 2-iminothiolane was diluted 10 mg/ml in borate buffer and added to 5 ml of the PLL at the following ratios:

| ml of PLL | ml of 2 iminothiolane | Estimated |
|---|---|---|
| at 10 mg/ml | (10 mg/ml) added | 2-iminothiolane:PLL Ratio |
| 5 | 0.22 | 5:1 |
| 5 | 0.09 | 2:1 |

The thiolation reaction was carried out at room temperature for 45 minutes. The thiolated poly-lysine agent (PLL(SH)ₙ was dialyzed against borate buffer for 4 hours with two buffer changes every two hours.

### Example 3

### Generation of Surface Modified Colloidal Gold Nanoparticles Using PEG Thiol

The method described by Horisberger (1979) was adapted to produce colloidal gold particles of various sizes. 250 ml of a 2% gold chloride solution (23.03% stock; OMG, South Plainfield, N.J.) was made in de-ionized H₂O (DIH₂O) and was heated to a rolling boil under reflux. The PEG(SH)ₙ was reconstituted to a concentration of 50 mg/ml in borate buffer. 1 or 2 ml of the PEG(SH)ₙ was added to the boiling gold chloride solution. The solution was boiled for an additional 45 minutes, cooled, and filtered though a 0.22μ filter. The sols were stored at room temperature until use.

### Example 4

### Generation of Surface Modified Colloidal Gold Nanoparticles Using Poly-L-Lysine Thiol

The method described by Horisberger (1979) was adapted to produce colloidal gold particles of various sizes. 250 ml of a 2% gold chloride solution (23.03% stock; OMG, South Plainfield, N.J.) was made in de-ionized H₂O (DIH₂O) and was heated to a rolling boil under reflux. The dialyzed PLL(SH)ₙ reagent was added directly to the gold solution without further dilution. The solution was boiled for an additional 45 minutes, cooled, and filtered though a 0.22μ filter. The sols were stored at room temperature until use.

### Example 5

### Determination of pH Binding Optimum

The binding of proteins to colloidal gold is known to be dependent on the pH of the colloid gold and protein solutions. The pH binding optimum of TNF to colloidal gold sols was empirically determined. This pH optimum was defined as the pH that allowed TNF to bind to the colloidal gold particle, but blocked salt-induced (by NaCl) precipitation of the particles. Naked colloidal gold particles are kept in suspension by their mutual electrostatic repulsion generated by a net negative charge on their surface. The cations present in a salt solution cause the negatively charged colloidal gold particles, which normally repel each other, to draw together. This aggregation/precipitation is marked by a visual change in the color of the colloidal gold solution from red to purple (as the particles draw together) and ultimately black, when the particles form large aggregates that ultimately fall out of solution. The binding of proteins or other stabilizing agents to the particles' surface block this salt-induced precipitation of the colloidal gold particles.

The pH optimum of TNF binding to colloidal gold was determined using 2 ml aliquots of 34 nm colloidal gold sol whose pH was adjusted from pH 5 to 11 (determined by using pH strips) with 1N NaOH. TNF (Knoll Pharmaceuticals; purified to homogeneity) was reconstituted in DIH₂O to a concentration of 1 mg/ml and further diluted to 100 μ/ml in 3 mM TRIS base. To determine the pH binding optimum for TNF, 100 μl of the 100 μg/ml TNF stock was added to the various aliquots of pH-adjusted colloidal gold. The TNF was incubated with the colloid for 15 minutes. Subsequently 100 μl of a 10% NaCl solution was added to each of the aliquots to induce particle precipitation. The optimal binding pH was defined as the pH, which allowed TNF to bind to the colloidal gold particles, while preventing the particles' precipitation by salt. While this description discloses a process by which to determine the pH binding optimum using the Frens preparation. It is contemplated by the instant invention that this method is also applicable for determining the pH binding optimum of the functionalized/reactive colloidal metal particles.

### Example 6

### Particle Characterization

Particle size was determined by differential centrifugal sedimentation using a DCS; disc centrifuge (CPS Instruments, Inc.). This technique measures particle size by determining the time required for the colloidal gold particles to traverse a sucrose density gradient created in a disc centrifuge. The DCS method uses calibrated particle reference standards to estimate the size of the colloidal gold preparation.

The size of the colloidal gold nanoparticles formed by the Frens reaction (Frens, Nature Phys. Sci., 241:20-22 1972) is determined by the amount of citrated added to the gold chloride solution. As the amount of citrate added to gold solution increases more gold nuclei are formed and as a result less free gold is available for particle growth. Consequently increasing the amount of citrate results in the formation of a greater number of particles of smaller diameter. Conversely, reducing the amount of citrate added leads to the formation of fewer gold nuclei that undergo particle growth, to form relatively large particles.

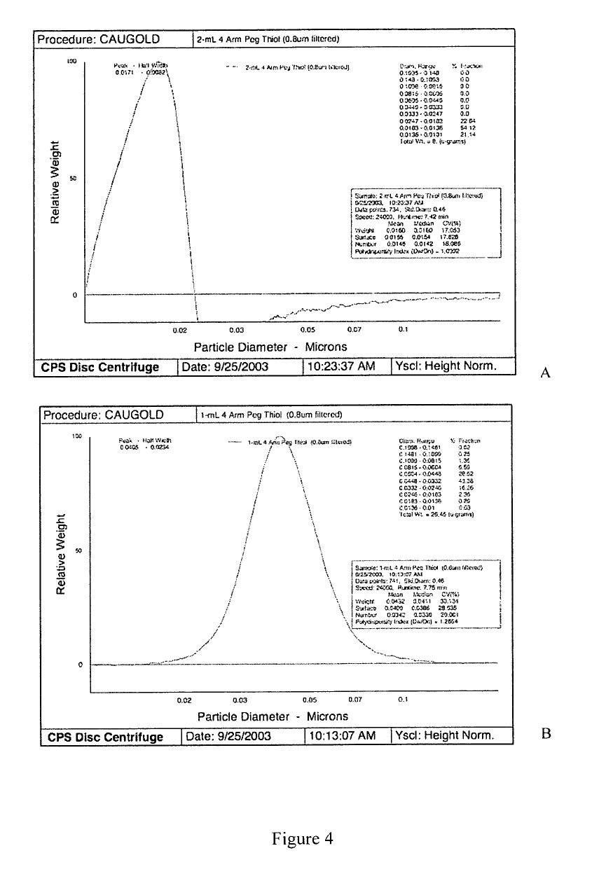

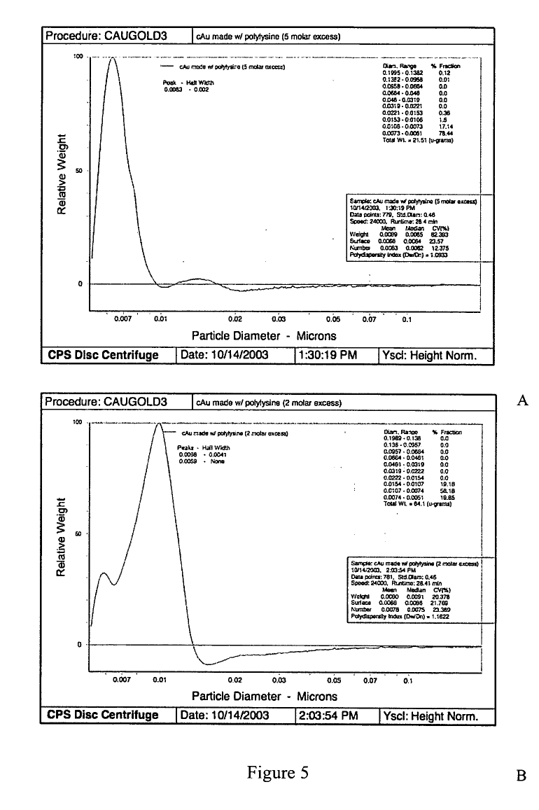

Particle sizing data, shown in FIGS. 4 and 5, reveals that as the amount of reducing agent is increased the size of the resultant particle decreases. The amount of thiol added was manipulated by two mechanisms. First, the physical mass of the functional reducing agent added to the make the particle was altered. This type of manipulation, performed herein with a representative reducing agent, the PEG(SH)₄ reagent, shows that doubling the number of thiol groups added to the gold chloride solution reduces the size of the particle from 42 to 16 nm (FIG. 4). A similar relationship is obtained using a representative reducing agent, the PLL(SH)₅ reducing agent. However unlike the PEG based reagent the mass of the thiol group used to reduce the gold chloride was manipulated by changing the number of thiol groups carried by a single polymer molecule. Thus, as shown in FIG. 5 although the amount of PLL(SH)₅ added to both reactions is the same the amount of reducing agent is significantly different. In essence these data show that as the number of thiol groups/unit polymer is increased the size of the colloidal gold particle formed is decreased.

### Example 7

### Binding Characteristics of Functionalized Particles

The TNF binding characteristics of the PLL(SH)ₙ and PEG(SH)ₙ functionalized/reactive particles generated by thiolated poly-1-lysine and PEG-thiol reduction, respectively, were compared to the nanoparticles generated by the Frens method (sodium citrate reduction). Previous studies revealed that TNF optimally binds to the Frens particle when the pH of the sol is adjusted between 8-9 (Paciotti, et al., Drug Delivery, 11:169-183, 2004). Thus for these studies the pH of 1 ml aliquots of the Frens, PLL(SH)ₙ or PEG(SH)ₙ functionalized/reactive particles were adjusted to 8 with NaOH. Subsequently 0.5-1.0 μg of TNF was added to the preparations. The samples were incubated for 15 minutes to allow TNF to bind to the particles. To separate particle bound TNF from free TNF the preparations were centrifuged for 15 minutes at 7500 rpms. After centrifugation a sample of the supernatant was collected and diluted in assay buffer. The remainder of the supematant was removed and the colloidal gold pellets were resuspended to their original volumes in assay buffer. The pellet and supernatant samples were serially diluted and analyzed by EIA (CytELISA TNF, CytImmune Sciences, Inc.).

Two binding studies were conducted with the PEG(SH)ₙ and PLL(SH)ₙ functionalized/reactive gold particle. Surprisingly, although both the PEG(SH)ₙ and PLL(SH)ₙ functionalized gold particles were stable against salt-induced precipitation, they differed with respect to TNF binding. Similar to the Frens preparation, the PEG(SH)ₙ functionalized/reactive gold particle bound a majority of the TNF added since little to no cytokine was present in the supernatant. This data suggest that the PEG(SH)ₙ polymer did not cover the entire surface of the particle and thus allowed TNF to bind. The data also suggest that the uncovered portions of the particles' surface are similar in nature to the surface of the particles in the Frens preparation. (See Table 1).

TABLE 1
TNF Binding on Frens and PLLSH functionalized colloidal gold
nanoparticles

| | Supernatant | Pellet | Total | Percent Free |
|---|---|---|---|---|
| PLL(SH)ₙ Functionalized | 836 | 13 | 849 | 98.4 |
| PEG(SH)ₙ Functionalized | 10 | 316 | 326 | 3.2 |
| Frens | 0.01 | 900 | 900 | 0.0 |

### Example 8

### DNA Binding of Poly-L-Lysine Particles

The functionality of the PLL(SH)ₙ functionalized/reactive preparation was examined by determining its ability to bind plasmid DNA. Previous work showed that native PLL, similar to salt solutions, causes a rapid agglomeration of the Frens preparation. However, the PLL present on the PLL(SH)ₙ functionalized particles serve to stabilize the particles in the presence of salt.

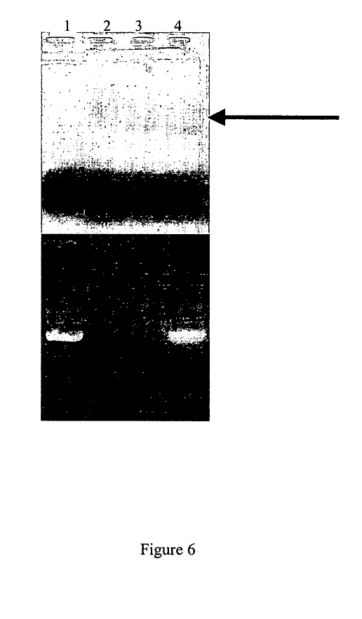

To determine whether thiolation could reverse the positive charge present on the amino groups, the thiolated poly-1-lysine polymer's ability to bind DNA was tested. The PLL(SH)ₙ-functionalized/reactive colloidal gold nanoparticles were incubated with 1 2 or 4 μg (lanes 2-4, respectively) of beta galactosidase plasmid DNA. Native DNA was used as a control. After a 15-minute incubation the samples were fractionated by agarose gel electrophoresis using a 1% gel. The co-migration of the PLL(SH)ₙ particles with the DNA was documented by photographing the gel under white and UV lighting. (FIG. 6)

These data suggest that the poly-1-lysine moiety covered a portion the particle surface to prevent the salt-induced precipitation.

While not wishing to be bound by any theory, the differential response observed between PEG(SH)ₙ and PLL(SH)ₙ functionalized/reactive colloidal particles and the binding of TNF is theorized to be the result of the surface charge of the functionalized/reactive particles. It is hypothesized that differently charged nanoparticles affect the ability of other charged agents to bind. It is believed that the PEG(SH)ₙ particles with a neutral or negative charge, do not inhibit the attraction and binding of the TNF to the PEG(SH)ₙ particle. In contrast, the positive charge of the PLL(SH)ₙ functionalized/reactive colloidal particles is theorized to repel, inhibit or prevent TNF from binding directly to the particle surface. Nevertheless, the thiolated poly-1-lysine particles were shown to bind plasmid DNA: a molecule that does not directly bind to typical colloidal gold particles (i.e., the Frens preparation).

### Example 9

### PEG-Thiol Vector for Tumor-Targeted Delivery of Antiangiogenic Drugs

These experiments used a PT-cAU-TNF-endostatin vector, a vector comprising two agents. It is thought that the TNF provided targeting functions for delivery of the therapeutic agent, endostatin (END), to the tumor. It is also theorized that once at the target, both agents may provide therapeutic effects. An aspect of the vector composition is the ratio of the targeting molecule, the therapeutic molecule and the PEG. All three entities are found on the same particle of colloidal gold.

The PT-cAu _{(TNF)}-END was made in three steps. First, TNF associated with the gold particles at a very low subsaturating mass of TNF. Unlike the PT-cAu-TNF vector, which was made with a concentration of TNF of 0.5 μg/ml, this vector was made with a TNF concentration of 0.05 μg/ml. TNF (diluted in 3 mM CAPS buffer, pH=10), which was added to the reagent bottle of the apparatus at a concentration of 0.1 μg/ml. The second bottle in the apparatus was filled with an equal volume of colloidal gold at a pH of 10. TNF was bound to the colloidal gold particles by activation of the peristaltic pump as previously described. The colloidal gold-TNF solution was incubated for 15 minutes and subsequently placed back into the gold container of the apparatus. The reagent bottle was then filled with an equal volume of endostatin (diluted in CAPS buffer at a concentration of 0.15 to 0.3 μg/ml. In an alternative embodiment, endostatin may be chemically modified by the addition of a sulfur group using agents such as n-succinimidyl-S-acetylthioacetate, to aid in binding to the gold particle.

The peristaltic pump was activated to draw the colloidal gold bound TNF and endostatin solutions into the T-connector. Upon complete interactions of the solutions the mixture was incubated in the collection bottle for an additional 15 minutes. The presence of additional binding sites for the PEG-Thiol was confirmed by the ability of salt to precipitate the particle at this stage. After the 15 minute incubation, 5K PEG-Thiol was added to the cAu_{(TNF)-}END vector and concentrated by diafiltration as previously described.

An alternative method for binding the two proteins to the same particle of gold comprises adding the agents simultaneously to the gold. TNF and END were placed in the reagent chamber of the binding apparatus. The concentration of each protein was 0.25 μg/ml and as a result, 1 ml of solution contained 0.5 μg of total protein. After binding the dual agent composition to gold particles, this colloidal gold preparation also precipitated in the presence of salt, indicating that additional free binding sites were available to bind the PEG-thiol. After a 15 minute incubation, 5K PEG-Thiol was added to the cAu_{(TNF)-}END vector and subsequently processed as described above.

After diafiltration, the retentate was measured for TNF and END concentrations in their respective EIA. To confirm the presence of END and TNF on the same particle of colloidal gold, a cross-antibody capture and detection assay was designed and used.

Samples of the PT-cAu_{(TNF)}-END vector were added to EIA plates coated with either the TNF or END capturing antibodies. The samples were incubated with the capturing antibody for 3 hours. After incubation the plates were washed and blotted dry. To bind any END present on a TNF captured sample, a biotinylated rabbit anti-endostatin polyclonal antibody was added to the wells. After a 30-minute incubation, the plates were washed and the presence of the biotinylated antibody was detected with streptavidin conjugated alkaline phosphatase. The generation of a positive color signal by the endostatin detection system indicated that the detection antibody bound to the chimeric vector previously captured by the TNF monoclonal antibody. By reversing the capturing and detection antibodies and using appropriate secondary detection systems, an assay was used to detect the presence of TNFα on an END-captured particle.

The data from these studies are presented in Table II. As can be seen in Table II, the retentate of the vector samples had 17 μg/ml of TNF and 22 μg/ml of END. These same samples also generated positive signals in the cross-antibody assays suggesting that both TNF and endostatin were on the same particle of colloidal gold.

While this description discloses a vector and method by which two agents are bound to a colloidal metal particle prepared by the Frens method. It is contemplated by the instant invention that this method is also suitable for the preparation of a vector composition wherein the colloidal metal particles are functionalized/reactive colloidal metal particles. In another embodiment, it is contemplated that the functionalized/reactive colloidal metal particles are formed by reducing agents, such as PEG(SH)ₙ or PLL(SH)ₙ. The resulting functionalized/reactive colloidal metal particles are then utilized in the method disclosed above to generate a two-agent bound functionalized/reactive colloidal metal particle.

TABLE II
The TNF and Endostatin concentrations present in retentates
of the PT-cAu_{(TNF)}-END vector.

| Sample | Analyte Tested | Concentration |
|---|---|---|
| PT-cAu_{(TNF)}-END | TNF | 17 μg/ml |
| | END | 22 μg/ml |

It must be noted that, as used in this specification and the claims, the singular forms “a,” “an,” and “the” include plural referents unless the context clearly dictates otherwise. Thus, for example, reference to a vector composition containing “an agent” means molar quantities of such an agent.

All patents, publications and abstracts cited above are hereby incorporated by reference in their entirety. U.S Provisional Application No. 60/540,075, filed Jan. 28, 2004 is hereby incorporated herein by reference. It is to be understood that this invention is not limited to the particular combinations, methods, and materials disclosed herein as such combinations, methods, and materials may vary somewhat. It is also to be understood that the terminology employed herein is used for the purpose of describing particular embodiments only and is not intended to be limiting.

1. A method for the production of functionalized nanoparticles comprising;
mixing a solution of a metal salt with a reducing agent to form a reaction mixture;
incubating the reaction mixture for a sufficient period of time to form functionalized nanoparticles.

2. The method of claim 1, wherein the metal salt comprises gold, silver, aluminum, ruthenium, zinc, iron, nickel or calcium.

3. The method of claim 1, wherein the reducing agent comprises derivatized polyethylene glycol, derivatized poly-amino acid, derivatized poly-1-lysine, PEG(SH)ₙ, or thiolated poly-1-lysine.

4. The method of claim 1, wherein the functionalized nanoparticle further comprises a chemical, therapeutic agent, pharmaceutical agent, drug, biological factor, enzyme, antigen, antibodies, proteins, lipids, nucleic acids, carbohydrates, nucleic acids, proteins, lipids, nutrients, cofactors, nutriceuticals, anesthetic or detection agents.

5. The method of claim 1, wherein the size of the functionalized nanoparticles comprises approximately 1 nm to about 100 nm in diameter.

6. A method for the production of a vector composition comprising;
mixing a solution of a metal salt with a reducing agent to form a first reaction mixture;
incubating the first reaction mixture for a sufficient period of time to form functionalized nanoparticles; and,
admixing the functionalized nanoparticles with at least one second agent to form a second reaction mixture;
incubating the second reaction mixture for a sufficient period of time to allow the functionalized nanoparticles to bind at least one second agent to form a vector composition; and,
isolating the vector composition from the second reaction mixture.

7. The method of claim 6, wherein the metal salt comprises gold, silver, aluminum, ruthenium, zinc, iron, nickel or calcium.

8. The method of claim 6, wherein the reducing agent comprises derivatized polyethylene glycol, derivatized poly-amino acid, derivatized poly-1-lysine, PEG(SH)ₙ, or thiolated poly-1-lysine.

9. The method of claim 6, wherein the second agent comprises a chemical agent, therapeutic agent, pharmaceutical agent, drug, biological factor, enzyme, antigen, antibodies, proteins, lipids, nucleic acids, carbohydrates, nucleic acids, proteins, lipids, nutrients, cofactors, nutriceuticals, anesthetic or detection agents.

10. The method of claim 6, wherein the size of the functionalized nanoparticles comprises approximately 1 nm to about 100 nm in diameter.

11. The method of claim 6, wherein the metal salt comprises gold, wherein the reducing agent comprises PEG-(SH)ₙ and wherein the second agent comprises tumor necrosis factor.

12. The method of claim 6, wherein the vector compositions is used to treat disease.

13. The method of claim 12, wherein a therapeutically effective amount of the vector composition is administered to a human or animal and wherein the disease comprises cancer, solid tumor cancer, Crohn's disease, breast cancer, Psoriasis, inflammatory bowel disease, adult respiratory distress syndrome, allergies, rhinitis, eczema, urticaria, anaphylaxis, transplant rejection, rheumatic diseases, systemic lupus erthymatosus, rheumatoid arthritis, seronegative spondyloarthritides, Sjogren's syndrome, systemic sclerosis, polymyositis, dermatomyositis, Type I Diabetes Mellitus, Acquired Immune Deficiency Syndrome, Hashimoto's thyroiditis, Graves' disease, Addison's disease, polyendocrine autoimmune disease, hepatitis, sclerosing cholangitis, primary biliary cirrhosis, pernicious anemia, coeliac disease, antibody-mediated nephritis, glomerulonephritis, Wegener's granulomatosis, microscopic polyarteritis, polyarteritis nodosa, pemphigus, dermatitis herpetiformis, psoriasis, vitiligo, multiple sclerosis, encephalomyelitis, Guillain-Barre syndrome, Myasthenia Gravis, Lambert-Eaton syndrome, sclera, episclera, uveitis, chronic mucocutaneous candidiasis, Bruton's syndrome, transient hypogammaglobulinemia of infancy, myeloma, X-linked hyper IgM syndrome, Wiskott-Aldrich syndrome, ataxia telangiectasia, autoimmune hemolytic anemia, autoimmune thrombocytopenia, autoimrune neutropenia, Waldenstrom's macroglobulinemia, amyloidosis, chronic lymphocytic leukemia, or non-Hodgkin's lymphoma.

14. The method of claim 12, wherein the functionalized nanoparticle comprises colloidal gold, wherein the second agent comprises tumor necrosis factor and wherein the disease comprises cancer.

15. A method for the production of functionalized nanoparticles comprising;
admixing a metal salt with a reducing agent to form a modified colloidal metal sol;
admixing at least one agent with a reducing agent to form a modified agent;
incubating the modified colloidal metal sol with the modified agent for a sufficient period of time to allow the modified colloidal metal sol to bind the modified agent forming functionalized nanoparticles.

16. The method of claim 12, wherein the reducing agent comprises comprises derivatized polyethylene glycol, derivatized poly-amino acid, derivatized poly-1-lysine, PEG(SH)ₙ, or thiolated poly-1-lysine.

17. The method of claim 12, wherein the second agent comprises a chemical agent, therapeutic agent, pharmaceutical agent, drug, biological factor, enzyme, antigen, antibodies, proteins, lipids, nucleic acids, carbohydrates, nucleic acids, proteins, lipids, nutrients, cofactors, nutriceuticals, anesthetic or detection agents.

18. A vector composition, comprising, a functionalized nanoparticle made by the process of mixing a solution of a metal salt with a reducing agent to form a reaction mixture;
incubating the reaction mixture for a sufficient period of time to form functionalized nanoparticles.

19. The vector composition of claim 18, further comprising at least one agent.

20. The vector composition of claim 18, wherein the reducing agent comprises a PEG derivative or a poly-1-lysine derivative.

21. The vector composition of claim 19, wherein the at least one agent is tumor necrosis factor.

22. The vector composition of claim 19, further comprising a targeting molecule, an integrating molecule, component-specific immunostimulating molecule, a chemical agent, therapeutic agent, pharmaceutical agent, drug, biological factor, enzyme, antigen, antibodies, proteins, lipids, nucleic acids, carbohydrates, nucleic acids, proteins, lipids, nutrients, cofactors, nutriceuticals, anesthetic or detection agents.

23. The vector composition of claim 22, wherein the component-specific immunostimulating molecule comprises Interleukin-1 (“IL-1”), Interleukin-2 (“IL-2”), Interleukin-3 (“IL-3”), Interleukin-4 (“IL-4”), Interleukin-5 (“IL-5”), Interleukin-6 (“IL-6”), Interleukin-7 (“IL-7”), Interleukin-8 (“IL-8”), Interleukin-10 (“IL-10”), Interleukin-11 (“IL-11”), Interleukin-12 (“IL-12”), Interleukin-13 (“IL-13”), lipid A, phospholipase A2, endotoxins, staphylococcal enterotoxin B and other toxins, Type I Interferon, Type II Interferon, Tumor Necrosis Factor (“TNF-α”), Transforming Growth Factor-β (“TGF-β”),) Lymphotoxin, Migration Inhibition Factor, Granulocyte-Macrophage Colony-Stimulating Factor (“CSF”), Monocyte-Macrophage CSF, Granulocyte CSF, vascular epithelial growth factor, Angiogenin, transforming growth factor (“TGF-α”), heat shock proteins, carbohydrate moieties of blood groups, Rh factors, fibroblast growth factor, inflammatory and immune regulatory proteins, nucleotides, DNA, RNA, mRNA, sense, antisense, cancer cell specific antigens; flt3 ligand/receptor system; B7 family of molecules and receptors; CD 40 ligand/receptor; immunotherapy drugs; angiogenic and anti-angiogenic drugs, basic fibroblast growth factor, or vascular endothelial growth factor (“VEGF”).
